(19) United States
(12) Patent Application Publication
(10) Pub. No.: US 2024/0242805 A1
(43) Pub. Date: Jul. 18, 2024

(54) SYSTEM AND METHOD FOR COMPLIANCE PREDICTION BASED ON DEVICE USAGE AND PATIENT DEMOGRAPHICS

(71) Applicant: ResMed Inc., San Diego, CA (US)

(72) Inventors: Grant Hilton DITTMER, San Diego, CA (US); Ryan Matnew HERNANDEZ, San Diego, CA (US); Prakhar SHUKLA, San Diego, CA (US); Kaushik GHOSHAL, San Diego, CA (US); Badri RAGHAVAN, San Diego, CA (US); Sourav Raj DEY, Oakland, CA (US); Jakov KUCAN, Oakland, CA (US); Michael STEFFERSON, Oakland, CA (US); Alexander Bruce NG, Oakland, CA (US); Jason Matthew CARPENTER, Oakland, CA (US); Alexander M. FIEDLER, Oakland, CA (US); John E. MAYNARD, Oakland, CA (US); Tameez SUNDERJI, Oakland, CA (US); Vivek MOHTA, Oakland, CA (US); Josh HAYES, Oakland, CA (US); Justin CHEN, Oakland, CA (US); Sam HITOV, Oakland, CA (US); Yuen Sang HO, Bedford, Nova Scotia (CA); Timothy HOFLER, San Diego, CA (US); Amar KOHLI, Bedford, Nova Scotia (CA); Sunghee WOO, San Diego, CA (US); Ian William Charles MYJER, Oakland, CA (US); Stanley Jackson Spencer BAKER, Oakland, CA (US); Zhao GAO, Oakland, CA (US); Manish LADHA, San Diego, CA (US)

(21) Appl. No.: 18/562,210

(22) PCT Filed: May 20, 2022
(86) PCT No.: PCT/US2022/030360

§ 371 Date: Nov. 17, 2023

Related U.S. Application Data
(60) Provisional application No. 63/191,235, filed on May 20, 2021.

(51) Int. Cl. G16H 20/00; G16H 10/60; G16H 40/67; G16H 50/20; G16H 50/70

(52) U.S. Cl. CPC G16H 20/00; G16H 10/60; G16H 40/67; G16H 50/20; G16H 50/70

(57) ABSTRACT

A system and method for providing compliance predictions for using a respiratory pressure therapy device in a treatment regimen is disclosed. The system includes a respiratory pressure therapy device having a transmitter and an air control device to provide respiratory therapy to a patient. The respiratory pressure therapy device collects operational data and transmits the collected operational data. Demographic data of the patient is collected. A predicted compliance with the treatment regimen is determined based on inputting operational data and demographic data to a machine learning compliance prediction model having a compliance prediction output. The machine learning model is trained from the operational data and demographic data of a population of patients using respiratory pressure therapy devices.

### PRIORITY CLAIM

The present disclosure claims priority to and the benefit of U.S. Provisional Patent Application Ser. No. 63/191,235 filed May 20, 2021. The contents of that application are hereby incorporated by reference in their entirety.

### TECHNICAL FIELD

The present disclosure relates generally to health data systems, and more specifically for a system that provides compliance predictions on data collected from respiratory pressure therapy devices and patient related data.

### BACKGROUND

Chronic diseases are the leading causes of death and disability worldwide. By 2020 their contribution is expected to rise to 73% of all deaths and 60% of the global burden of disease. This is associated with soaring costs of health care. Chronic diseases are the primary driver of health care costs, accounting for ninety cents (90¢) of every dollar spent in the U.S. This cost is related to an aging population. In 2015, one out of eight people worldwide was aged 60 years or over. By 2030, it is estimated that one in six people will be aged 60 years or over.

Many patients suffering from chronic disease require supplemental oxygen as part of long-term oxygen therapy (LTOT). Currently, the vast majority of patients that are receiving LTOT are diagnosed under the general category of Chronic Obstructive Pulmonary Disease (COPD). This general diagnosis includes such common diseases as Chronic Bronchitis, Emphysema, and related pulmonary conditions. Other patients may also require supplemental oxygen, for example, obese patients who need to maintain elevated activity levels, patients with cystic fibrosis or infants with broncho-pulmonary dysplasia.

Many such patients utilize respiratory pressure therapy devices to assist in treating respiratory or sleep ailments. For example, CPAP devices provide air pressure when needed to keep air passageways open during sleep. Such devices may be used to treat a range of respiratory disorders. Examples of respiratory disorders include Periodic Limb Movement Disorder (PLMD), Restless Leg Syndrome (RLS), Sleep-Disordered Breathing (SDB), Obstructive Sleep Apnea (OSA), Respiratory Effort Related Arousal (RERA), Central Sleep Apnea (CSA), Cheyne-Stokes Respiration (CSR), respiratory insufficiency, Obesity Hyperventilation Syndrome (OHS), Chronic Obstructive Pulmonary Disease (COPD), Neuromuscular Disease (NMD), and chest wall disorders. These disorders are often treated using a respiratory therapy system. Certain disorders may be characterized by particular events, e.g., apneas, hypopncas, and hyperpneas.

The operation of a CPAP therapy device relies on continuous positive airway pressure acting as a pneumatic splint and preventing upper airway occlusion, such as by pushing the soft palate and tongue forward and away from the posterior oropharyngeal wall. However, treatment of sleep apnea by CPAP therapy devices is generally voluntary, and hence patients may elect not to comply with therapy if they find devices used to provide such therapy uncomfortable, difficult to use, expensive, or aesthetically unappealing.

Due to the difficulties of user compliance, such CPAP devices are configured to transmit certain operational data during operation by the user. Such data allows determination of whether the patient prescribed with respiratory therapy has been “compliant”, e.g., that the patient has used their respiratory pressure therapy device according to one or more “compliance rules.” One example of a compliance rule for CPAP therapy is that a patient, in order to be deemed compliant, is required to use the respiratory pressure therapy device for at least four hours a night for at least twenty-one (21) of thirty (30) consecutive days in a ninety (90) day period. In order to determine a patient's compliance, a provider of the respiratory pressure therapy device, such as a health care provider, may manually obtain data describing the patient's therapy using the respiratory pressure therapy device. The provider may calculate the usage over a predetermined time period, and compare it with the compliance rule. Once the health care provider has determined that the patient has used their respiratory pressure therapy device according to the compliance rule, the health care provider may notify a third party, such as a payor, that the patient is compliant.

Patients often use their CPAP devices regularly upon first receiving the devices, but fail to continue consistently using the devices such that the patients are considered compliant. Of these non-compliant patients, 17.7% came within 5 days of achieving compliance. Currently, patient device data cannot be effectively used to determine when patients are at risk of becoming non-compliant, because the existing compliance process is a ‘fix after problem’ approach powered by rules largely based on patient device data. These rules are static, do not learn over time, have limited personalization, and require manual configuration and management even with action groups. In addition, the current process cannot scale to analyze large numbers of patients because patients are not prioritized and are equally grouped by issue. Healthcare providers and other actors are therefore forced to make subjective predictions as to which patients may be non-compliant in the future. Such predictions are unreliable, as they may vary based on the different judgments of different actors and such actors do not know and/or do not have relevant data for such predictions.

In order to increase compliance, there is a trend toward technology solutions for older people with a higher prevalence of sleep-disordered breathing and co-morbid conditions. Such solutions may include smartphone applications, continuous positive airway pressure (CPAP) usage applications, wearable activity monitors, Internet of Medical things (IoMT), artificial intelligence (AI), and communication technologies such as Wi-Fi, Bluetooth, 4G, and 5G.

However, current chronic disease management treatments and associated assistance applications tend to have poor results as the user has to regularly interact with them and may provide inaccurate information to the application. This is especially the case if the user believes certain answers may change the price/cost or availability of insurance or care. In other cases, a user simply cannot judge if their condition is worsening or not, or which symptoms are more important than others.

There is a large group of patients who trend toward compliance but ultimately do not reach compliance, and many of these patients come very close to compliance. Providers have limited resources/time and need to prioritize patients who may be approaching non-compliance, but who would respond positively to additional resources such as coaching and outreach, or adjustments to device settings, and achieve compliance. Thus, it would be advantageous to quickly and efficiently prioritize resources, adjustments to settings, and other interventions based on how likely a patient is to becoming non-compliant. There is therefore a need for a system that may predict compliance of a patient for using a respiratory pressure treatment device.

### SUMMARY

One disclosed example is a method of predicting compliance with a respiratory treatment regimen for a patient. Operational data is collected from a respiratory pressure therapy device. Demographic data of the patient is collected. Compliance is predicted with the treatment based on inputting operational data and demographic data to a machine learning compliance prediction model with a compliance prediction output. The machine learning model is trained from the operational data and demographic data of a population of patients using respiratory pressure therapy devices.

A further implementation of the example method is where the method also includes sending the compliance data to a user device operated by a health care provider associated with the patient. Another implementation is where the example method includes sending the compliance data to a user device operated by the patient. Another implementation is where the example method includes providing motivation to the patient via the user device based on the compliance prediction. Another implementation is where the example method includes classifying the patient based on a plurality of classifications of the population of patients. Another implementation is where the machine learning compliance prediction model includes an output based on the classification of the patient. Another implementation is where the machine learning compliance prediction model outputs the prediction each day of the treatment regimen having a predetermined period of time. Another implementation is where the example method includes communicating with the respiratory pressure therapy device to change the respiratory therapy to the patient in response to the compliance prediction. Another implementation is where the respiratory pressure therapy device includes a motor, a blower, and a mask to supply pressurized air to the patient. The collected operational data includes data from a flow rate sensor, an air pressure sensor, and a motor speed transducer. Another implementation is where the respiratory pressure therapy device is one of a positive airway pressure (PAP) device, a non-invasive ventilation (NIV) device, or an adaptive support ventilation (ASV) device. Another implementation is where the example method includes collecting physiological data from a health monitor. The collected physiological data is input to the machine learning compliance prediction model to determine the prediction. Another implementation is where the health monitor includes at least one sensor, the at least one sensor selected from one of the group of an audio sensor, a heart rate sensor, a respiratory sensor, a ECG sensor, a photoplethysmography (PPG) sensor, an infrared sensor, an activity sensor, a radio frequency sensor, a SONAR sensor, an optical sensor, a doppler radar motion sensor, a thermometer, an impedance sensor, a piezoelectric sensor, a photoelectric sensor, or a strain gauge sensor. Another implementation is where the example method includes receiving collected operational data via a user device and transmitting the data via a network. Another implementation is where the machine learning compliance prediction model analyzes environmental data related to the patient in determining the prediction. Another implementation is where the machine learning compliance prediction model analyzes demographic data related to the patient in determining the prediction. Another implementation is where the example method includes providing a shap value for each type of usage data and demographic data. Another implementation is where the collected operation data is used to derive duration of usage, leak data, AHI, and mask type. Another implementation is where the demographic data includes age and gender. Another implementation is where the example method includes collecting patient input data from a survey. The machine learning compliance prediction model analyzes the patient input data in determining the prediction. Another implementation is where the method includes displaying the compliance prediction of the patient. Another implementation is where the compliance prediction is displayed in a calendar showing the period of treatment. Another implementation is where the calendar shows past days of compliance. Another implementation is where the calendar shows the end of a projected compliance period based on the compliance prediction. Another implementation is where the method includes displaying information relating to a last contact attempt with the patient. Another implementation is where the display includes contact data for the patient.

Another disclosed example is a computer program product comprising instructions which, when executed by a computer, cause the computer to carry out the above methods. Another implementation of the computer program product is where the computer program product is a non-transitory computer readable medium.

Another disclosed example is a system to predict compliance of a patient with a respiratory treatment regimen. The system includes a respiratory pressure therapy device having a transmitter and an air control device to provide air flow based respiratory therapy to a patient. The respiratory pressure therapy device collects operational data and transmits the collected operational data. A patient database stores demographic data associated with the patient. A network receives the collected operational data from the respiratory pressure therapy device and the demographic data from the database. A compliance analysis engine is coupled to the network. The compliance analysis engine inputs the collected operational data and demographic data to a compliance prediction model trained on a large patient population dataset including operational and demographic data and resulting compliance. The compliance prediction model outputs a prediction of compliance for the patient for using the respiratory pressure therapy device.

A further implementation of the example system includes a network interface coupled to the compliance analysis engine. The network interface sends the compliance data to a user device operated by a health care provider associated with the patient. Another implementation of the example system includes a network interface coupled to the compliance analysis engine. The network interface sends the compliance data to a user device operated by the patient. Another implementation is where the user device is configured to provide motivation to the patient based on the compliance prediction. Another implementation is where the compliance analysis engine classifies the patient based on a plurality of classifications of the population of patients. Another implementation is where the machine learning compliance prediction model includes an output based on the classification of the patient. Another implementation is where the machine learning compliance prediction model outputs the prediction each day of the treatment regimen having a predetermined period of time. Another implementation is where the compliance analysis engine is configured to communicate with the respiratory pressure therapy device to change the respiratory therapy to the patient in response to the compliance prediction. Another implementation is where the respiratory pressure therapy device includes a motor, a blower, and a mask to supply pressurized air to the patient. The collected operational data includes data from a flow rate sensor, an air pressure sensor, and a motor speed transducer. Another implementation is where the respiratory pressure therapy device is one of a positive airway pressure (PAP) device, a non-invasive ventilation (NIV) device, or an adaptive support ventilation (ASV) device. Another implementation is where the system includes a health monitor. The compliance analysis engine is configured to collect physiological data from the health monitor. The collected physiological data is input to the machine learning compliance prediction model to determine the prediction. Another implementation is where the health monitor includes at least one sensor, the at least one sensor selected from one of the group of an audio sensor, a heart rate sensor, a respiratory sensor, a ECG sensor, a photoplethysmography (PPG) sensor, an infrared sensor, an activity sensor, a radio frequency sensor, a SONAR sensor, an optical sensor, a doppler radar motion sensor, a thermometer, an impedance sensor, a piezoelectric sensor, a photoelectric sensor, or a strain gauge sensor. Another implementation is where the system includes a user device configured to receive the collected operational data and transmit the data via a network. Another implementation is where the machine learning compliance prediction model analyzes environmental data related to the patient in determining the prediction. Another implementation is where the machine learning compliance prediction model analyzes demographic data related to the patient in determining the prediction. Another implementation is where the machine learning compliance prediction model provides a shap value for each type of usage data and demographic data. Another implementation is where the collected operation data is used to derive duration of usage, leak data, AHI, and mask type. Another implementation is where the demographic data includes age and gender of the patient. Another implementation is where the compliance analysis engine collects patient input data from a survey. The machine learning compliance prediction model analyzes the patient input data in determining the prediction. Another implementation is where the system includes a display is coupled to the compliance analysis engine. The display displays the compliance prediction of the patient. Another implementation is where the compliance prediction is displayed in a calendar showing the period of treatment. Another implementation is where the calendar shows past days of compliance. Another implementation is where the calendar shows the end of a projected compliance period based on the compliance prediction. Another implementation is where the display displays information relating to a last contact attempt with the patient. Another implementation is where the display includes contact data for the patient.

Another disclosed example is a method of training a compliance prediction model for outputting a prediction of compliance for a patient with a respiratory treatment regimen. Operational data is collected from a population of patients each using a respiratory pressure therapy device. Demographic data is collected from the population of patients. Compliance data is determined from the population of patients based on the operational data. A set of input features is selected based on the collected operational and demographic data. A compliance prediction model is trained with the collected operational and demographic data selected in the set of input features and the corresponding compliance data to output a compliance prediction.

A further implementation of the example method includes collecting environmental data related to the population of patients as one of the set of input features. Another implementation is where the example method includes training the compliance prediction model to provide a shap value for each of the set of input features. Another implementation is where the set of input features includes duration of usage, leak data, AHI, and mask type. Another implementation is where the demographic data includes age and gender. Another implementation is where the method includes collecting patient input data from a survey as one of the set of input features.

The above summary is not intended to represent each embodiment or every aspect of the present disclosure. Rather, the foregoing summary merely provides an example of some of the novel aspects and features set forth herein. The above features and advantages, and other features and advantages of the present disclosure will be readily apparent from the following detailed description of representative embodiments and modes for carrying out the present invention when taken in connection with the accompanying drawings and the appended claims.

### BRIEF DESCRIPTION OF THE DRAWINGS

The disclosure will be better understood from the following description of exemplary embodiments together with reference to the accompanying drawings, in which:

FIG. 1 is a block diagram of an example health analysis system that collects data from a respiratory pressure therapy device used by a patient;

FIG. 2A shows a respiratory pressure therapy device in accordance with one form of the present technology;

FIG. 2B is a schematic diagram of the pneumatic path of a respiratory pressure therapy device in accordance with one form of the present technology;

FIG. 2C is a schematic diagram of the electrical components of a respiratory pressure therapy device in accordance with one form of the present technology;

FIG. 3 is an example data collection system that allows for training of a machine learning model for compliance prediction;

FIG. 4A is a block diagram of collection of data for training a machine learning model for compliance prediction;

FIG. 4B is a flow diagram of the machine learning model for compliance prediction;

FIG. 5A is a block diagram of the process of providing predictions from the machine learning model in the system in FIG. 3;

FIG. 5B is a block diagram of the data processed by a compliance prediction algorithm using the machine learning model in FIG. 5A;

FIG. 6A is an example image of an interface generated by an application using prediction data from the prediction system;

FIG. 6B is a screen image of the example interface in FIG. 6A when a specific search function is used;

FIG. 6C is a screen image of the example interface in FIG. 6A when a patient details search function is used;

FIG. 6D is a screen image of the example interface in FIG. 6A when a days in therapy search function is used;

FIG. 6E is a screen image of the example interface in FIG. 6A when a days in compliance search function is used;

FIG. 6F is a screen image of the example interface in FIG. 6A when a compliance search function is used;

FIG. 6G is a screen image of the example interface in FIG. 6A when a usage data search function is used;

FIG. 7A is an example of a compliance graph showing compliance prediction results for a one day to compliance in a 90-day cycle;

FIG. 7B is an example of a compliance graph showing compliance prediction results for a four day to compliance in a 90-day cycle;

FIG. 8A is a screen image of an alternative patient information interface generated by an application;

FIG. 8B is a screen image of an alternative interface generated by an application using prediction data with a calendar function displayed from a detailed patient window;

FIG. 8C is a screen image of therapy graphs displayed from the detailed patient window;

FIG. 8D is a screen image of a timeline of patient events displayed from the detailed patient window;

FIG. 9A is a screen image of a patient notes popup;

FIG. 9B is a screen image of a patient review popup; and

FIG. 10 is a flow diagram of the process of collecting patient data for determining a prediction for compliance.

The present disclosure is susceptible to various modifications and alternative forms. Some representative embodiments have been shown by way of example, in the drawings and will be described in detail herein. It should be understood, however, that the invention is not intended to be limited to the particular forms disclosed. Rather, the disclosure is to cover all modifications, equivalents, and alternatives falling within the spirit and scope of the invention as defined by the appended claims.

### DETAILED DESCRIPTION OF THE ILLUSTRATED EMBODIMENTS

The present inventions can be embodied in many different forms. Representative embodiments are shown in the drawings, and will herein be described in detail. The present disclosure is an example or illustration of the principles of the present disclosure, and is not intended to limit the broad aspects of the disclosure to the embodiments illustrated. To that extent, elements and limitations that are disclosed, for example, in the Abstract, Summary, and Detailed Description sections, but not explicitly set forth in the claims, should not be incorporated into the claims, singly or collectively, by implication, inference, or otherwise. For purposes of the present detailed description, unless specifically disclaimed, the singular includes the plural and vice versa; and the word “including” means “including without limitation.” Moreover, words of approximation, such as “about,” “almost,” “substantially,” “approximately,” and the like, can be used herein to mean “at,” “near,” or “nearly at,” or “within 3-5% of,” or “within acceptable manufacturing tolerances,” or any logical combination thereof, for example.

The present disclosure relates to a system that generates predictive machine learning models which are continuously evolving, learning, and are personalized to identify and prioritize patients who may be approaching non-compliance with a respiratory treatment plan or regimen requiring operation of home medical equipment (HME), such as a respiratory pressure therapy device. The prediction system may be embedded in a home medical equipment facing web application that may be operated by a healthcare provider for diagnosis, monitoring, compliance and therapy management in relation to respiratory therapy devices and patients. One example of such an application is AirView™ provided by ResMed, which is targeted for use by HME front-line staff who are responsible for patient outreach and coaching in the use of such home medical equipment. With a limited number of staff and hours in a day, the compliance predictions produced by the system allows HME staff to focus their efforts on those patients are who at risk of non-compliance in a more efficient way and more easily identify that particular cohort of patients.

The prediction may be based partly on usage data from usage of a respiratory pressure therapy device. An example respiratory pressure therapy device may monitor and collect operational data such as the motor voltage, RPM, air flow, and mask leak. The example respiratory pressure therapy device may also monitor and collect physiological data via cardiac signals derived from a microphone in or near the tube, from the mask signal, from an optical sensor near or on the face (such as in the mask), from electrodes (in or on the mask, headgear), from a connected patch with electrical or optical sensing, or from a smart watch, bracelet, or ring. Data could also be integrated from other devices, such as a smart inhaler, used by the patient. Additional data related to the patient is also input into the prediction system. The operational data from the equipment and the patient data is input to the machine learning model to provide a compliance prediction for the user of the respiratory pressure therapy device.

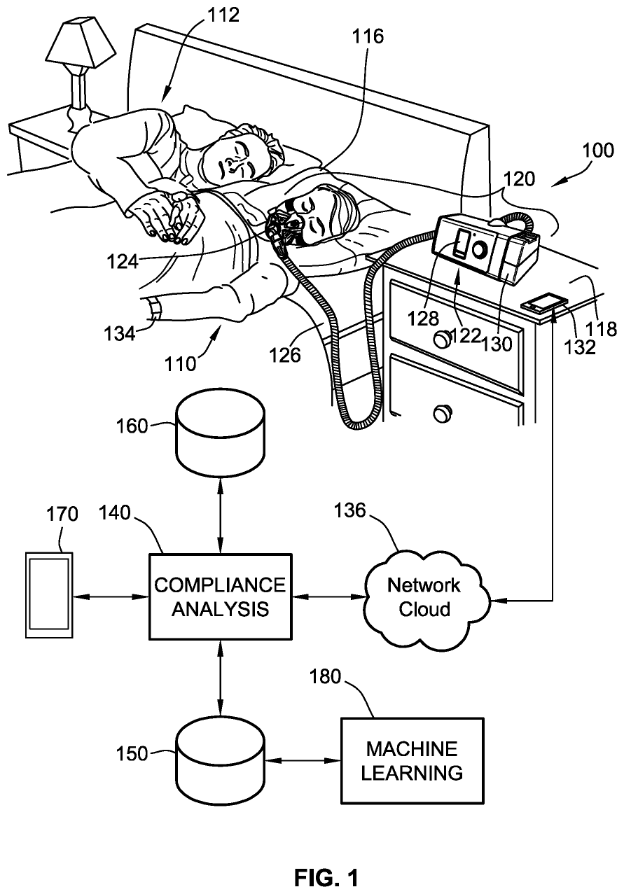

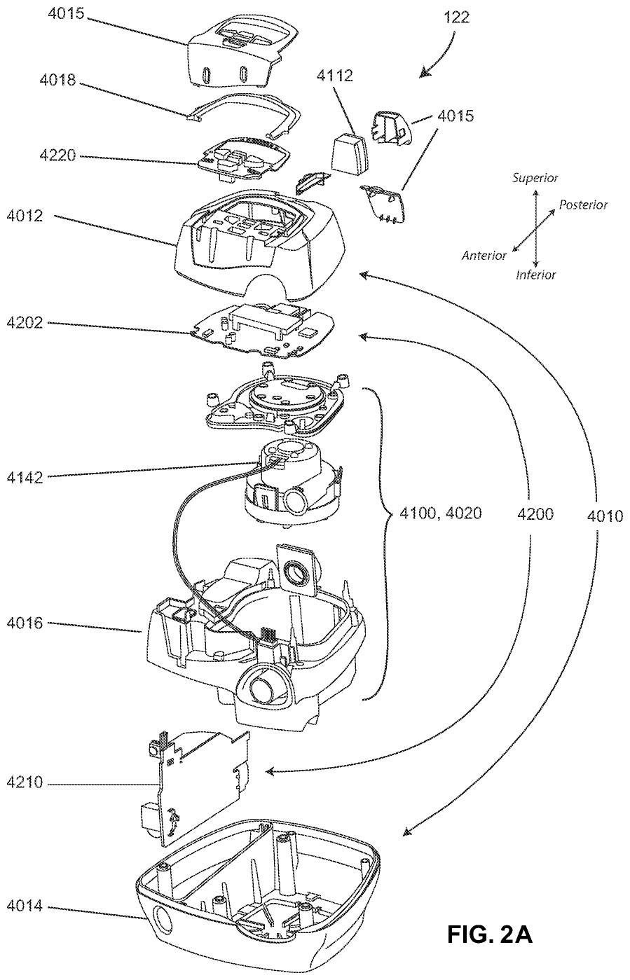

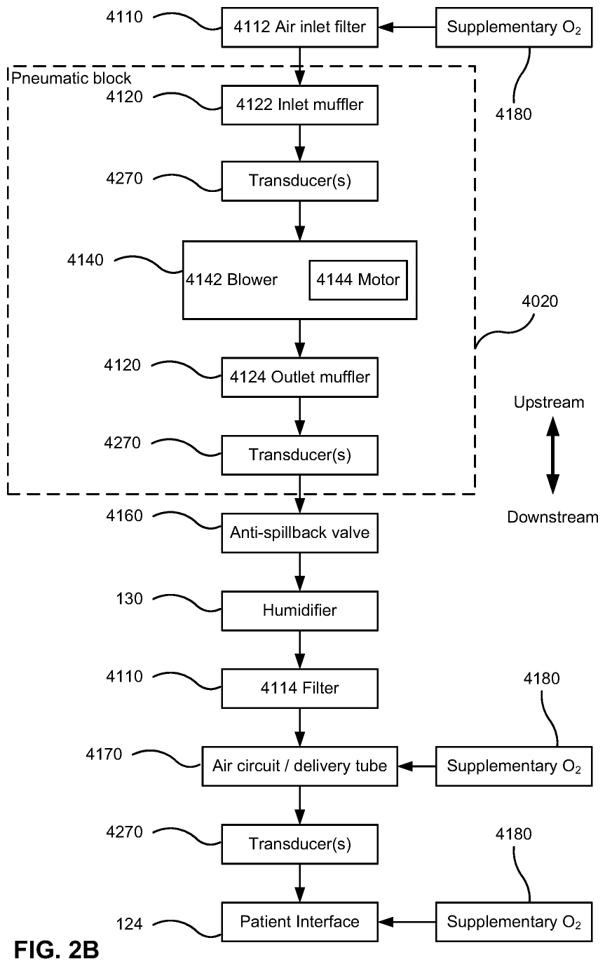

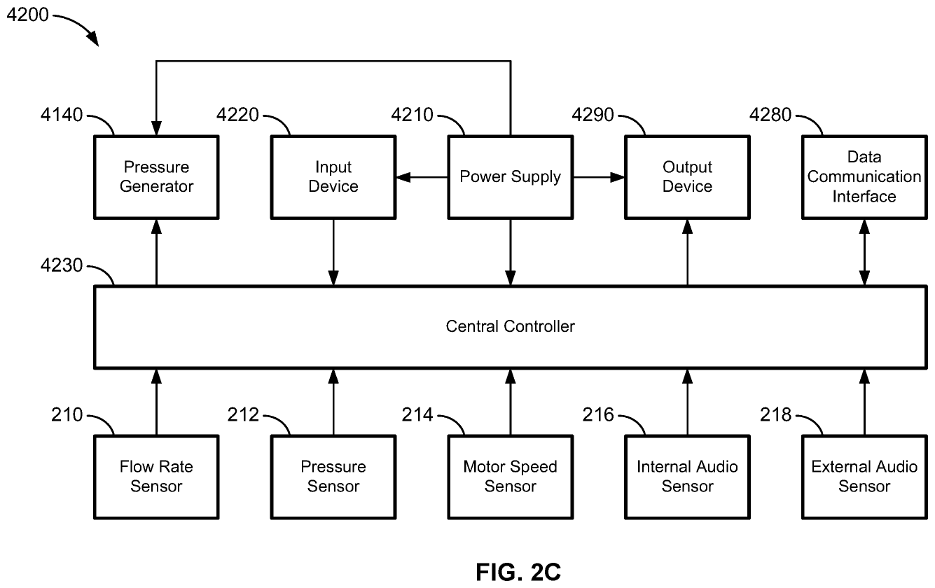

FIG. 1 shows a respiratory treatment and monitoring system 100 for a patient 110 wearing a user interface, in the form of a mask, receiving a supply of air at positive pressure from a respiratory pressure therapy (RPT) system 120. The patient 110 (a user of the respiratory system 120) and a bed partner 112 are located in a bed 116 and are laying on a mattress. A user interface 124 (e.g., a full face mask) can be worn by the patient 110 during a sleep session. The user interface 124 is fluidly coupled and/or connected to a respiratory pressure therapy (RPT) device 122 via a conduit 126. In turn, the respiratory device 122 delivers pressurized air to the patient 110 via the conduit 126 and the user interface 124 to increase the air pressure in the throat of the patient 110 to aid in preventing the airway from closing and/or narrowing during sleep. The respiratory device 122 can be positioned on a nightstand 118 that is directly adjacent to the bed 116 as shown in FIG. 2, or more generally, on any surface or structure that is generally adjacent to the bed 116 and/or the patient 110.

The respiratory system 120 can include the respiratory pressure therapy device 122 (referred to herein as respiratory device 122), the user interface 124, the conduit 126 (also referred to as a tube or an air circuit), a display device 128, a humidification tank 130, or any combination thereof. In some implementations, the respiratory device 122 can include a memory device, one or more sensors, and the humidification tank 130. Respiratory pressure therapy refers to the application of a supply of air to an entrance to a user's airways at a controlled target pressure that is nominally positive with respect to atmosphere throughout the user's breathing cycle (e.g., in contrast to negative pressure therapies such as the tank ventilator or cuirass). The respiratory system 120 is generally used to treat individuals suffering from one or more sleep-related respiratory disorders (e.g., obstructive sleep apnea, central sleep apnea, or mixed sleep apnea).

The respiratory device 122 is generally used to generate pressurized air that is delivered to a user (e.g., using one or more motors that drive one or more compressors). In some implementations, the respiratory device 122 generates continuous constant air pressure that is delivered to the user. In other implementations, the respiratory device 122 generates two or more predetermined pressures (e.g., a first predetermined air pressure and a second predetermined air pressure). In still other implementations, the respiratory device 122 is configured to generate a variety of different air pressures within a predetermined range. For example, the respiratory device 122 can deliver at least about 6 cm H₂O, at least about 10 cm H₂O, at least about 20 cm H₂O, between about 6 cm H₂O and about 10 cm H₂O, between about 7 cm H₂O and about 12 cm H₂O, etc. The respiratory device 122 can also deliver pressurized air at a predetermined flow rate between, for example, about −20 L/min and about 150 L/min, while maintaining a positive pressure (relative to the ambient pressure).

The user interface 124 engages a portion of the user's face and delivers pressurized air from the respiratory device 122 to the user's airway to aid in preventing the airway from narrowing and/or collapsing during sleep. This may also increase the user's oxygen intake during sleep. Depending upon the therapy to be applied, the user interface 124 may form a seal, for example, with a region or portion of the user's face, to facilitate the delivery of gas at a pressure at sufficient variance with ambient pressure to effect therapy, for example, at a positive pressure of about 10 cm H₂O relative to ambient pressure. For other forms of therapy, such as the delivery of oxygen, the user interface may not include a seal sufficient to facilitate delivery to the airways of a supply of gas at a positive pressure of about 10 cm H₂O.

In some implementations, the user interface 124 is a face mask that covers the nose and mouth of the user. Alternatively, the user interface 124 can be a nasal mask that provides air to the nose of the user or a nasal pillow mask that delivers air directly to the nostrils of the user. The user interface 124 can include a plurality of straps (e.g., including hook and loop fasteners) for positioning and/or stabilizing the interface on a portion of the user (e.g., the face) and a conformal cushion (e.g., silicone, plastic, foam, etc.) that aids in providing an air-tight seal between the user interface 124 and the user. In some examples, the user interface 124 can be a tube-up mask, wherein straps of the mask are configured to act as conduit(s) to deliver pressurized air to the face or nasal mask. The user interface 124 can also include one or more vents for permitting the escape of carbon dioxide and other gases exhaled by the patient 110. In other implementations, the user interface 124 can comprise a mouthpiece (e.g., a night guard mouthpiece molded to conform to the user's teeth, a mandibular repositioning device, etc.).

The conduit 126 (also referred to as an air circuit or tube) allows the flow of air between two components of a respiratory system 120, such as the respiratory device 122 and the user interface 124. In some implementations, there can be separate limbs of the conduit for inhalation and exhalation. In other implementations, a single limb conduit is used for both inhalation and exhalation.

One or more of the respiratory pressure therapy device 122, the user interface 124, the conduit 126, the display device 128, and the humidification tank 130 can contain one or more sensors (e.g., a pressure sensor, a flow rate sensor, or more generally any of the other sensors described herein). These one or more sensors can be use, for example, to measure the air pressure and/or flow rate of pressurized air supplied by the respiratory device 122.

The display device 128 is generally used to display image(s) including still images, video images, or both and/or information regarding the respiratory device 122. For example, the display device 128 can provide information regarding the status of the respiratory device 122 (e.g., whether the respiratory device 122 is on/off, the pressure of the air being delivered by the respiratory device 122, the temperature of the air being delivered by the respiratory device 122, etc.) and/or other information (e.g., a sleep score or a therapy score (such as a myAir™ score), the current date/time, personal information for the patient 110, etc.). In some implementations, the display device 128 acts as a human-machine interface (HMI) that includes a graphic user interface (GUI) configured to display the image(s) as an input interface. The display device 128 can be an LED display, an OLED display, an LCD display, or the like. The input interface can be, for example, a touchscreen or touch-sensitive substrate, a mouse, a keyboard, or any sensor system configured to sense inputs made by a human user interacting with the respiratory device 122.

The humidification tank 130 is coupled to or integrated in the respiratory device 122 and includes a reservoir of water that can be used to humidify the pressurized air delivered from the respiratory device 122. The respiratory pressure therapy device 122 can include a heater to heat the water in the humidification tank 130 in order to humidify the pressurized air provided to the user. Additionally, in some implementations, the conduit 126 can also include a heating element (e.g., coupled to and/or imbedded in the conduit 126) that heats the pressurized air delivered to the user.

The respiratory system 120 can be used, for example, as a ventilator or a positive airway pressure (PAP) system such as a continuous positive airway pressure (CPAP) system, an automatic positive airway pressure system (APAP), a bi-level or variable positive airway pressure system (BPAP or VPAP), or any combination thereof. The CPAP system delivers a predetermined air pressure (e.g., determined by a sleep physician) to the user. The APAP system automatically varies the air pressure delivered to the user based on, for example, respiration data associated with the user. The BPAP or VPAP system is configured to deliver a first predetermined pressure (e.g., an inspiratory positive airway pressure or IPAP) and a second predetermined pressure (e.g., an expiratory positive airway pressure or EPAP) that is lower than the first predetermined pressure.

Generally, a user who is prescribed usage of a respiratory system will tend to experience higher quality sleep and less fatigue during the day after using the respiratory system 120 during the sleep compared to not using the respiratory system 120 (especially when the user suffers from sleep apnea or other sleep related disorders). However, many users do not conform to their prescribed usage because the user interface 124 is uncomfortable or cumbersome, or due to other side effects (e.g., dry eyes, dry throat, noise, etc.). Users are more likely to fail to use the respiratory system 120 as prescribed (or discontinue usage altogether) if they fail to perceive that they are experiencing any benefits (e.g., less fatigue during the day).

While the respiratory therapy system 120 described herein is an example of a therapy system, other types of therapy systems for aiding in treating sleep-related disorders are contemplated for compliance predictions. Other therapy systems can include an oral appliance, such as, for example, a dental appliance or mandibular repositioning device (MRD). Oral appliance therapy can help prevent the collapse of the tongue and soft tissues in the back of the throat by supporting the jaw (mandible) in a forward position, keeping the user's airway open during sleep.

The system 100 allows for monitoring respiratory related data for the patient 110. The system 100 thus includes an optional external device such as a user device 132, and a body mounted health monitoring device 134. The user device 132 and possibly the monitoring device 134 and the respiratory device 122 may be in communication with a network 136. The system 100 also includes a remote cloud-based compliance analysis engine 140. The compliance analysis engine 140 may run on a networked computing device or devices available through the cloud. The analysis engine 140 may be a cloud-based system in this example that is coupled via the network 136. The compliance analysis engine 140 thus is in network communication with the respiratory pressure therapy device 122. The user device 132 may operate applications that assist the patient 110 in operation of the RPT device 122 and facilitate compliance. One example of such an application is the MyAir™ application offered by ResMed. A patient assistance application thus automatically sends patient sleep data in the form of a daily score (e.g., a MyAir™ score) to any web-accessible device chosen by the patient. By allowing patients to track their nightly sleep data and through tailored coaching, the application empowers patients to stay engaged with therapy to assist in compliance. The patient application may interface with the RPT device 122 and display compliance related operational data such as usage hours, mask leaks, events based on apnea-hypopnea index (AHI) and mask on/mask off events. The patient application may also display surveys to collect patient input data that may be used for the machine learning model prediction of compliance. For example, surveys may ask the patient about how sleepy they are after therapy and how well they are doing so far with their therapy. These responses and others can form new input features added to the machine learning model.

The health monitoring device 134 is generally used to aid in generating physiological data for determining an activity measurement associated with the user. The body-mounted health monitoring device 134 may be smart wearable clothing, smart watch, or a smart device, in order to capture data in a low impact manner continuously from the patient 110. The activity measurement can include, for example, a number of steps, a distance traveled, a number of steps climbed, a duration of physical activity, a type of physical activity, an intensity of physical activity, time spent standing, a respiration rate, an average respiration rate, a resting respiration rate, a maximum respiration heart rate, a respiration rate variability, a heart rate, an average heart rate, a resting heart rate, a maximum heart rate, a heart rate variability, a number of calories burned, blood oxygen saturation, electrodermal activity (also known as skin conductance or galvanic skin response), or any combination thereof. The health monitoring device 134 includes one or more of the sensors described herein, such as, for example, an audio sensor, a heart rate sensor, a respiratory sensor, an ECG sensor, a photoplethysmography (PPG) sensor, an infrared sensor, an activity sensor, a radio frequency sensor, a SONAR sensor, an optical sensor, doppler radar motion sensors, a thermometer, or impedance, piezoelectric, photoelectric, or strain gauge type sensors. This data can be fused with other data sources collected during the day or data collected during certain periods of time, such as from operating the RPT device 122.

In some implementations, the health monitoring device 134 is a wearable device that can be worn by the user, such as a smartwatch, a wristband, a ring, or a patch. For example, referring to FIG. 1, the health monitoring device 134 is worn on a wrist of the patient 110. The health monitoring device 134 can also be coupled to or integrated a garment or clothing that is worn by the user. Alternatively still, the health monitoring device 134 can also be coupled to or integrated in (e.g., within the same housing) the user device 132. More generally, the health monitoring device 134 can be communicatively coupled with, or physically integrated in (e.g., within a housing), the respiratory system 120, and/or the user device 132.

The respiratory pressure therapy device 122 includes a transmitter and an air control device to provide respiratory therapy to the patient 110. As will be explained, the respiratory pressure therapy device 122 collects operational and usage data and transmits the collected usage data and operational data to the remote compliance analysis engine 140. The compliance analysis engine 140 receives the collected data from the respiratory therapy device 122 to determine a prediction as to compliance to the patient 110 using the respiratory therapy device 122. The prediction is also predicated on patient specific data. Thus, the engine 140 may also receive and add other relevant data from a patient information database 150, the health monitoring device 134, and the mobile device 132. External databases, such as a database 160 may also provide additional data for the analysis of the health condition. For example, the database 160 may include “big data” from other respiratory pressure therapy devices and corresponding patients. The database 160 may also store relevant external data from other sources such as environmental data, scientific data, and demographic data. External devices such as a workstation or a mobile user device 170, accessible by a healthcare provider, may be connected to the compliance analysis engine 140, as will be explained below. In this example, the healthcare provider may access applications through the mobile user device 170 that provide diagnosis, compliance and therapy management in relation to data received from the RPT device 122 of patients associated with the health care provider. An example of such an application is the AirView™ application.

The user devices 132 and 170 each include a display. The user devices 132 and 170 can be, for example, a mobile device such as a smart phone, a tablet, a laptop, or the like. Alternatively, the user devices 132 and 170 can be an external sensing system, a television (e.g., a smart television) or another smart home device (e.g., a smart speaker(s) such as Google Home, Amazon Echo, Alexa etc.). In some implementations, the user device is a wearable device (e.g., a smart watch). The displays on the devices 132 and 170 are generally used to display image(s) including still images, video images, or both. In some implementations, the display acts as a human-machine interface (HMI) that includes a graphic user interface (GUI) configured to display the image(s) and an input interface. The display can be an LED display, an OLED display, an LCD display, or the like. The input interface can be, for example, a touchscreen or touch-sensitive substrate, a mouse, a keyboard, or any sensor system configured to sense inputs made by a human user interacting with the user devices 132 and 170. In some implementations, one or more user devices can be used by and/or included in the system 100.

In some implementations, the database 150 stores a profile associated with the patient 110. The profile can include, for example, demographic information associated with the patient, biometric information associated with the patient, medical information associated with the patient, self-reported patient feedback, sleep parameters associated with the patient (e.g., sleep-related parameters recorded from one or more earlier sleep sessions), or any combination thereof. The demographic information can include, for example, information indicative of an age of the patient, a gender of the patient, a race of the patient, a geographic location of the patient, a relationship status, a family history of insomnia, an employment status of the patient, an educational status of the patient, a socioeconomic status of the patient, or any combination thereof. The medical information can include, for example, including indicative of one or more medical conditions associated with the patient, medication usage by the patient, or both. The medical information data can further include a multiple sleep latency test (MSLT) test result or score and/or a Pittsburgh Sleep Quality Index (PSQI) score or value. The self-reported patient feedback can include information indicative of a self-reported subjective sleep score (e.g., poor, average, excellent), a self-reported subjective stress level of the patient, a self-reported subjective fatigue level of the patient, a self-reported subjective health status of the patient, a recent life event experienced by the user, or any combination thereof.

Data from the database 150 and health conditions may be further correlated by a machine-learning engine 180. The machine-learning engine 180 may implement machine-learning structures such as a neural network, decision tree ensemble, support vector machine, Bayesian network, or gradient boosting machine. Such structures can be configured to implement either linear or non-linear predictive models for determining different health conditions. For example, data processing such as compliance prediction may be carried out by any one or more of supervised machine learning, deep learning, a convolutional neural network, and a recurrent neural network. In addition to descriptive and predictive supervised machine learning with hand-crafted features, it is possible to implement deep learning on the machine-learning engine 180. This typically relies on a larger amount of scored (labeled) data (such as many hundreds of nights of scored sleep and health data from different RPT devices) for normal and abnormal conditions for different classifications of patients. This approach may implement many interconnected layers of neurons to form a neural network (“deeper” than a simple neural network), such that more and more complex features are “learned” by each layer. Machine learning can use many more variables than hand-crafted features or simple decision trees.

Convolutional neural networks (CNNs) are used widely in audio and image processing for inferring information (such as for face recognition), and can also be applied to audio spectrograms, or even population scale genomic data sets created from the collected data in the system 100 represented as images. When carrying out image or spectrogram processing, the system cognitively “learns” temporal and frequency properties from intensity, spectral, and statistical estimates of the digitized image or spectrogram data.

In contrast to CNNs, not all problems can be represented as one with fixed-length inputs and outputs. For example, processing respiratory sounds or acoustic sounds of the heart has similarities with speech recognition and time series prediction. Thus, the sound analysis can benefit from a system to store and use context information such as recurrent neural networks (RNNs) that can take the previous output or hidden states as inputs. In other words, they may be multilayered neural networks that can store information in context nodes. RNNs allow for processing of variable length inputs and outputs by maintaining state information across time steps, and may include LSTMs (long short term memories, types of “neurons” to enable RNNs increased control over, which can be unidirectional or bidirectional) to manage the vanishing gradient problem and/or by using gradient clipping.

The machine-learning engine 180 may be trained for supervised learning of known usage and patient data from known data inputs for assistance in analyzing input data. The training includes compliance data specific to patients that may be derived from the usage data. The machine-learning engine 180 may also be trained for unsupervised learning to determine unknown correlations between input data and compliance, to increase the range of compliance analysis of the analysis engine 140.

In this example, the RPT device 122 may include electronic components to act as a communications hub to manage data transfer with other sensors in the vicinity of the patient 110, and transfer of the collected data for remote processing by the compliance analysis engine 140. Example of other sensors may include blood pressure monitors, weighing scales, sleep sensors, blood glucose monitors, and smart drug/medication adherence bins. Such data may be collected by the RPT device 122 even when the RPT device 122 is not delivering therapy itself. Alternatively, the user device 132 may collect data from the health monitoring device 134, the RPT device 122, and other data sources, and thus serve as a communications hub to manage data transfer to remote processing to the compliance analysis engine 140. Other devices such as home digital assistants that may communicate with the RPT device 122 may also serve as the communications hub.

FIG. 2A shows an exploded view of the components of the example RPT device 122 in accordance with one aspect of the present technology comprises mechanical, pneumatic, and/or electrical components and is configured to execute one or more algorithms, such as any of the methods, in whole or in part, described herein. FIG. 2B shows a block diagram of the example RPT device 122. FIG. 2C shows a block diagram of the electrical control components of the example RPT device 122. The directions of upstream and downstream are indicated with reference to the blower and the patient interface. The blower is defined to be upstream of the patient interface, and the patient interface is defined to be downstream of the blower, regardless of the actual flow direction at any particular moment. Items which are located within the pneumatic path between the blower and the patient interface are downstream of the blower and upstream of the patient interface. The RPT device 122 may be configured to generate a flow of air for delivery to a patient's airways, such as to treat one or more of the respiratory conditions described elsewhere in the present document.

The RPT device 122 may have an external housing 4010, formed in two parts, an upper portion 4012 and a lower portion 4014. Furthermore, the external housing 4010 may include one or more panel(s) 4015. The RPT device 122 comprises a chassis 4016 that supports one or more internal components of the RPT device 122. The RPT device 122 may include a handle 4018.

The pneumatic path of the RPT device 122 may comprise one or more air path items, e.g., an inlet air filter 4112, an inlet muffler 4122, a pressure generator 4140 capable of supplying air at positive pressure (e.g., a blower 4142), an outlet muffler 4124, and one or more transducers 4270, that may be pressure sensors and flow rate sensors.

One or more of the air path items may be located within a removable unitary structure which will be referred to as a pneumatic block 4020. The pneumatic block 4020 may be located within the external housing 4010. In one form a pneumatic block 4020 is supported by, or formed as part of the chassis 4016.

The RPT device 122 may have an electrical power supply 4210, one or more input devices 4220, a central controller 4230, a therapy device controller 4240, a pressure generator 4140, one or more protection circuits 4250, transducers 4270, data communication interface 4280, and one or more output devices 4290. A separate controller may be provided for the therapy device. Electrical components 4200 may be mounted on a single printed circuit board assembly (PCBA) 4202. In an alternative form, the RPT device 122 may include more than one PCBA 4202. Other components such as the one or more protection circuits 4250, transducers 4270, the data communication interface 4280, and storage devices may also be mounted on the PCBA 4202.

An RPT device may comprise one or more of the following components in an integral unit. In an alternative form, one or more of the following components may be located as respective separate units.

The RPT device 122 in accordance with one form of the present technology may include an air filter 4110, or a plurality of air filters 4110. In one form, an inlet air filter 4112 is located at the beginning of the pneumatic path upstream of a pressure generator 4140. In one form, an outlet air filter 4114, for example, an antibacterial filter, is located between an outlet of the pneumatic block 4020 and a patient interface 3000.

The RPT device 122 in accordance with one form of the present technology may include a muffler 4120, or a plurality of mufflers 4120. In one form of the present technology, an inlet muffler 4122 is located in the pneumatic path upstream of a pressure generator 4140. In one form of the present technology, an outlet muffler 4124 is located in the pneumatic path between the pressure generator 4140 and the patient interface 124 in FIG. 1.

In one form of the present technology, a pressure generator 4140 for producing a flow, or supply, of air at positive pressure is a controllable blower 4142. For example, the blower 4142 may include a brushless DC motor 4144 with one or more impellers. The impellers may be located in a volute. The blower may be capable of delivering a supply of air, for example at a rate of up to about 120 liters/minute, at a positive pressure in a range from about 4 cm H2O to about 20 cm H2O, or in other forms up to about 30 cm H2O. The blower may be as described in any one of the following patents or patent applications the contents of which are incorporated herein by reference in their entirety: U.S. Pat. Nos. 7,866,944; 8,638,014; 8,636,479; and PCT Patent Application Publication No. WO 2013/020167.

The pressure generator 4140 is under the control of the therapy device controller 4240. In other forms, a pressure generator 4140 may be a piston-driven pump, a pressure regulator connected to a high pressure source (e.g., compressed air reservoir), or a bellows.

Transducers may be internal of the RPT device 122, or external of the RPT device 122. External transducers may be located, for example, on or form part of the air circuit, e.g., the patient interface 124. External transducers may be in the form of non-contact sensors such as a Doppler radar movement sensor that transmit or transfer data to the RPT device 122. In one form of the present technology, one or more transducers 4270 are located upstream and/or downstream of the pressure generator 4140. The one or more transducers 4270 may be constructed and arranged to generate signals representing properties of the flow of air such as a flow rate, a pressure or a temperature at that point in the pneumatic path. In one form of the present technology, one or more transducers 4270 may be located proximate to the patient interface 124.

In one form of the present technology, an anti-spill back valve 4160 is located between the humidifier 130 and the pneumatic block 4020. The anti-spill back valve is constructed and arranged to reduce the risk that water will flow upstream from the humidifier 130, for example to the motor 4144.

A power supply 4210 may be located internal or external of the external housing 4010 of the RPT device 122. In one form of the present technology, power supply 4210 provides electrical power to the RPT device 122 only. In another form of the present technology, power supply 4210 provides electrical power to both RPT device 122 and humidifier 130.

Internal sensors such as a flow rate sensor 210, a pressure sensor 212, and a motor speed transducer 214 may be coupled to the central controller 4230 in FIG. 2C. An optional internal audio sensor 216 may be embedded in the interface 124 in FIG. 1 to detect specific patient air sounds. An optional external audio sensor 218 such as a microphone may be located on the exterior of the RPT device 122, the interface 124, or the humidifier 130 to collect additional audio data. Additional sensors such as a heart rate sensor, an ECG sensor (providing cardiac fiducial parameters, of which peaks could be processed to estimate heart rate, detect arrhythmias and so forth), a pulse oximeter (SpO2) sensor (providing heart rate, oxygen saturation, and potential an estimate of blood pressure from pulse transit time), a blood pressure sensor, a room-temperature sensor, a contact or non-contact body temperature sensor, a room humidity sensor, a proximity sensor, a gesture sensor, a touch sensor, a gas sensor, an air quality sensor, a particulate sensor, an accelerometer, a gyroscope, a tilt sensor, other acoustic sensors such as passive or active SONAR, an ultrasonic sensor, a radio frequency sensor, an accelerometer, a light intensity sensor, a LIDAR sensor, an infrared sensor (passive, transmissive, or reflective), carbon dioxide sensor, a carbon monoxide sensor, or a chemical sensor, may be connected to the central controller 4230 via an external port. Data from such additional sensors may also be collected by the central controller 4230. Data from the sensors 210, 212, 214, 216, and 218 may be collected by central controller 4230 on a periodic basis. Such data generally relates to the operational state of the RPT device 122.

The controller 4230 may collect the data at different rates. For example, during normal use, the data may be collected at a low-resolution rate. As will be explained, a triggering event may cause the controller 4230 to start collecting the data at a different collection rate, such as at a higher resolution rate for collecting more data and/or additional types of data in a comparative time period than the low-resolution rate for more detailed analysis of the patient 110. In this example, the central controller 4230 encodes such data from the sensors in a proprietary data format. The data may also be coded in a standardized data format.

In one form of the present technology, the RPT device 122 includes one or more input devices 4220 in the form of buttons, switches, or dials to allow a person to interact with the device. The buttons, switches, or dials may be physical devices, or software devices accessible via a touch screen. The buttons, switches, or dials may, in one form, be physically connected to the external housing 4010, or may, in another form, be in wireless communication with a receiver that is in electrical connection to the central controller 4230. In one form, the input device 4220 may be constructed and arranged to allow a person to select a value and/or a menu option.

In one form of the present technology, the central controller 4230 is one or a plurality of processors suitable to control the RPT device 122. Suitable processors may include an x86 INTEL processor, a processor based on ARM® Cortex®-M processor from ARM Holdings such as an STM32 series microcontroller from ST MICROELECTRONIC. In certain alternative forms of the present technology, a 32-bit RISC CPU, such as an STR9 series microcontroller from ST MICROELECTRONICS or a 16-bit RISC CPU such as a processor from the MSP430 family of microcontrollers, manufactured by TEXAS INSTRUMENTS may also be suitable. In one form of the present technology, the central controller 4230 is a dedicated electronic circuit. In one form, the central controller 4230 is an application-specific integrated circuit. In another form, the central controller 4230 comprises discrete electronic components. The central controller 4230 may be configured to receive input signal(s) from one or more transducers 4270, one or more input devices 4220, and the humidifier 130.

The central controller 4230 may be configured to provide output signal(s) to one or more of an output device 4290, a therapy device controller 4240, a data communication interface 4280, and the humidifier 130.

In some forms of the present technology, the central controller 4230 is configured to implement the one or more methodologies described herein, such as the one or more algorithms expressed as computer programs stored in a non-transitory computer-readable storage medium, on an internal memory. In some forms of the present technology, the central controller 4230 may be integrated with an RPT device 122. However, in some forms of the present technology, some methodologies may be performed by a remotely located device such as the user device 132 in FIG. 1. For example, the remotely located device may determine control settings for a ventilator or detect respiratory-related events by analysis of stored data such as from any of the sensors described herein. As explained above, all data and operations for external sources or the central controller 4230 are generally proprietary to the manufacturer of the RPT device 122. Thus, the data from the sensors and any other additional operational data is not generally accessible by any other device.

In one form of the present technology, a data communication interface is provided, and is connected to the central controller 4230. The data communication interface may be connectable to a remote external communication network and/or a local external communication network. The remote external communication network such as the cloud 136 may be connectable to remote external devices such as servers or databases, as shown in FIG. 1. The local external communication network may be connectable to a local external device such as the mobile device 132 or the health monitoring device 134 in FIG. 1. Thus, the local external communication network may be used by either the RPT device 122 or the mobile device 132 to collect data from other devices.

In one form, the data communication interface is part of the central controller 4230. In another form, the data communication interface 4280 is separate from the central controller 4230, and may comprise an integrated circuit or a processor. In one form, the remote external communication network is the Internet. The data communication interface may use wired communication (e.g., via Ethernet, or optical fiber) or a wireless protocol (e.g., CDMA, GSM, 2G, 3G, 4G/LTE, LTE Cat-M, NB-IOT, 5G New Radio, satellite, beyond 5G) to connect to the Internet. In one form, local external communication network 4284 utilizes one or more communication standards, such as Bluetooth, or a consumer infrared protocol.

The example RPT device 122 includes integrated sensors and communication electronics, as shown in FIG. 2C. Older RPT devices may be retrofitted with a sensor module that may include communication electronics for transmitting collected data. Such a sensor module could be attached to the older RPT device and thus transmit operational data to the remote compliance analysis engine 140.

In this example, the system 100 may change the “resolution of data” that is uploaded from a PAP device such as the RPT device 122 to the cloud 136, the edge of the cloud, etc., based on an event and/or for a specific purpose that requires greater detail in the collected data. These smart health insights into co-morbidities with high resolution data and sensing can be enabled by utilizing the contact the RPT device 122 has with the patient 110 throughout the night (mask/pillows). This allows a change from traditional low resolution breath analysis to high resolution cardio-respiratory insights, augmented by cloud processing from the compliance analysis engine 140.

In this example, the RPT device 122 may output air flow and pressure signals at a relatively low frequency, such as 25 Hz. Other low-resolution data such as mask pressure, air pressure, EPR pressure, leak data, respiratory rate, tidal volume, minute ventilation, snoring, and flow limitation may be collected every two (2) seconds. Optional external sensors that may be connected to the RPT device 122 may allow other data such as oxygenation (SpO2) and pulse rate to be collected. In addition, standard annotations for exchange and storage of medical data, such as the European Data Format (EDF), may be applied to the collected data. As will be explained, the above data may be collected at a higher resolution mode. In addition, the high resolution mode may allow the collection of additional types of data or data derived from the basic data.

The flow data, motor speed, pressure and acoustic data from the example RPT device 122 may be used to profile the characteristics of the RPT device 122. As explained above, the sensors may be used to detect the flow generation from the motor, characteristics from the respiratory apparatus conduit such as the tube or hose to the mask, the mask or cannula fit, motor speed, and gas volumetric flow rate and outlet pressure (from a pressure transducer or a flow sensor). For example, this data may be used to identify accessories and parts such as mask type, tube or hose, number of hoses, and connectors. Data may be collected to determine any leaks that are indicative if the fit of the mask interface 124 is within acceptable parameters. As will be explained, leaks are a potential feature for machine learning input that influences the probability of compliance. This may also be correlated to whether a patient is using a mask of the best size or type, or whether the mask specifically must be replaced due to ordinary wear and tear. The tube may act as an acoustic waveguide from the internal acoustic sensor 216 in FIG. 2C. Data may be collected by the flow generator being run at a constant speed (to have largely flat sound data signal) or have a change introduced at a specific time.

The raw acoustic data from the acoustic sensor 216 may be processed in order to determine the transfer function of the tube, mask, and respiratory system to gain new insights about the condition of the equipment, the lungs of the patient 110, and gas composition. The transfer function may also be used to detect patient movement. For example, the reflection from the motor sound may show a peak relating to the round trip time from the acoustic sensor 216 to the mask cavity, and back. The system has knowledge of the tube length, and the speed of sound for various gases (such as typical composition of air, inhaled air, and changed composition when exhaled carbon dioxide is greater than the base CO2 level in air, or indeed any expected changes due to supplemental oxygen, changes in speed of sound of different gases at different temperatures, and humidity settings).

One approach to model the transfer function by calculating the impulse response function is using “Cepstrum” analysis. Cepstrum analysis has origins in analyzing seismic events and is useful in processing echo signals. Cepstrum analysis can allow separating different effects, as different events can be additive in the logarithm of the power spectrum and separable. Other processing approaches could include using a logarithmic power spectrum (LogFFT), a Segmented Chirp Z-Transform (SCZT) together with a flexible window, or time-frequency representations (TFRs) such the Short-Time Fourier Transform (STFT), Gabor expansion, the Cross-Ambiguity Function (CAF), and quadratic TFRs such as the Wigner-Ville Distribution (WVD). When considering Cepstral analysis, the autocepstrum is the ceptstrum of the autocorrelation of the signal. Cepstrum analysis involves the processing of sound samples from the tube, carrying out a Fourier transform, taking the Logarithm, and then an Inverse Fourier transform. The purpose of the process is to convert the convolution of an impulse response function (IRF) and a noise or sound signal into an addition operation to more easily allow the separation of the IRF for analysis.

The collected data may also be used to determine an analysis of sleep stage. For example, the movement of the patient interface, such as the mask interface 124, may be determined from an embedded motion sensor to determine movement of the patient. Normalized respiration rate variability and inspiration/expiration ratio changes may be determined for sleep stage analysis. Heart rate variability trends may also be determined for sleep stage analysis. For example, by tracking peaks in the cardiogenic signal seen in one or more of the flow, pressure, or microphone signals, the peak-to-peak time can be used to estimate number of heart beats per minute (HR bpm) and well as heart rate variability (such as spectral analysis of interbeat times). Such data may be correlated to how sleep architecture is evolving and used to determine whether the patient is proceeding through the expected number of sleep stages, whether the patient is sleep fragmented, whether the patient is receiving adequate REM and deep sleep, and whether the patient is going to the toilet for frequent urination. Estimated movement intensity, duration, and patterns of activity, heart rate, heart rate variability, breathing rate, breathing rate variability, normalized breathing rate variability, breathing signal waveform shape (inspiration, expiration pauses) may be calculated as part of sleep staging analysis system. In some embodiments, the flow and/or microphone signals may be supplied directly to the model, such as deep learning neural network system. The system may calculate wakefulness (conscious and unconscious micro-awakenings), light sleep (stages NREM N1, N2), N3 deep sleep/slow wave sleep, and REM sleep. The system learns changes in breathing rate and or heart rate variability during different stages of sleep, and the relationship with movements of the patient. Knowledge of sleep stage patterns can be used to adapt the therapy settings in order to optimize deep and REM sleep, maximize overall sleep time, and reduce awakenings and light sleep. Demographic data such as age and gender information for may be input into a machine learning model to determine the sleep stage. Inputs may include age, gender, estimated movement intensity, duration, and patterns of activity, heart rate, heart rate variability, breathing rate, breathing rate variability, normalized breathing rate variability, and breathing signal waveform shape (inspiration, expiration pauses) to a sleep staging analysis blocks for the machine learning model.

The sleep stage analysis may also be combined with other data to determine stress, mental health issues, or other physiological issues. For example, during a sleep stage, a drop in heart rate and blood pressure may be expected. If the collected data indicates a deviation from the expected drops, this provides an indication of stress, mental health issues, or other physiological issues. Another example may be predicting the likelihood of complex sleep apnea before commencing continuous positive airway pressure. This can be related to the severity of diagnosed sleep-disordered breathing before treatment, the prevalence of central events during that test, and continuing monitoring of central and obstructive events (and examples of periodic breathing) after diagnoses but prior to treatment (such as via acoustic or RF sensing or other movement and breathing estimator of sleep). Analysis of the sleep hypnogram/sleep architecture, particularly disruption/fragmentation, and the ratio of the hypopnea density in NREM versus REM sleep can also be used as an input feature. Appropriate therapy may then be selected to address the condition.

The collected data may be analyzed in the context of specific patient conditions that may be derived from data from other sources. Such data may include data input through an application running on the mobile device 132, or input from electronic health records on a database such as the database 160 in FIG. 1. The patient-specific data may therefore include conditions a patient may have such as pre-existing issues, demographic details (BMI, age, gender), and geographic details (allergen risks due to pollen count, heat exhaustion due to outside temperature, air quality and oxygen quantity due to altitude), and medications associated with the patient.

The patient input data may include subjective feedback on how the patient is feeling, whether the patient feels fatigued, and the level of sleepiness. The data may be used to determine the quality of sleep for the patient. This may be a comparison to a personal baseline for the patient, linked to weather data, a comparison to an average sleeper of their age and gender (aiming to be better than average), or a comparison to an average of a person with the same chronic conditions and or disease progression. As explained above, the baseline may be one determined from a normative patient population relative to the patient. Such data may be displayed in an application executed by the mobile device 132. Such data may also be made available on the user device 170 for a health care professional. The above described data may be collected and input to a machine learning model for generating an output to predict compliance by a patient in relation to use of the RPT device.

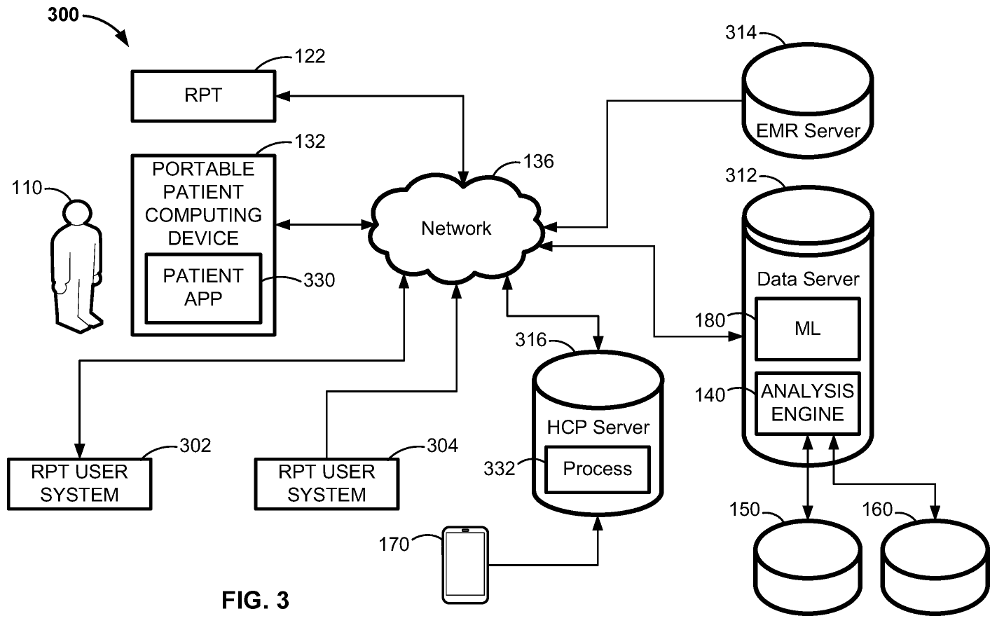

FIG. 3 is a block diagram of an example data collection system 300 that collects operational and usage data from RPT devices such as the RPT device 122 used by the patient 110 in FIG. 1 in a general patient population. A health care system may include multiple patients. Each of the patients may be using an RPT device with functionality similar to the RPT device 122, additional sensors, such as the health monitoring device 134, and associated mobile computing devices. Collectively, the RPT device, sensors, and mobile computing device for different patients in the patient population may be shown as RPT systems 302 and 304. Such systems collect data from the corresponding patients requiring respiratory therapy for the data collection system 300. The underlying health care system includes a data server 312, an electronic medical records (EMR) server 314, and a health or home care provider (HCP) server 316. In this example, the data server 312 is hardware that executes the compliance analysis engine 140 and has access to different databases, such as the patient information database 150 and other databases such as the database 160 for other relevant data for both training a compliance prediction machine learning model as well as providing predictions from the trained models. The patient database 150 thus may include compliance data related to compliance by patients with the respiratory therapy regimen using the RPT device.

These entities are all connected to, and configured to communicate with each other over the wide area network 136 such as the cloud or Internet. The connections to the wide area network 136 may be wired or wireless. The data server 312, EMR server 314, the HCP server 316, and the support server 318 may all be implemented on distinct computing devices at separate locations, or any sub-combination of two or more of those entities may be co-implemented on the same computing device.

The servers 312, 314, 316, and 318 are all a computer or network of computers. Although a simplified example is illustrated in FIG. 3, typically the application servers will be a server class system that uses powerful processors, large memory, and faster network components compared to a typical computing system used. The server typically has large secondary storage, for example, using a RAID (redundant array of independent disks) array and/or by establishing a relationship with an independent content delivery network (CDN) contracted to store, exchange and transmit data such as the asthma notifications contemplated above. Additionally, the computing system includes an operating system, for example, a UNIX operating system, LINUX operating system, or a WINDOWS operating system. The operating system manages the hardware and software resources of the application server and also provides various services, for example, process management, input/output of data, management of peripheral devices, and so on. The operating system provides various functions for managing files stored on a device, for example, creating a new file, moving or copying files, transferring files to a remote system, and so on.

The application server includes a software architecture for supporting access and use of the compliance engine 140 by many different devices through the network 136, and thus at a high level can be generally characterized as a cloud-based system. Generally, the application server is designed to handle a wide variety of data. The application server includes logical routines that perform a variety of functions including checking the validity of the incoming data, parsing and formatting the data if necessary, passing the processed data to a database for storage, and confirming that the database has been updated.

In this example, either the RPT device 122 or the mobile device 132 associated with the patient is configured to intermediate between the patient 110 and the remotely located entities of the health care system over the wide area network 136. In the implementation of FIG. 3, this intermediation is accomplished by a software application program 330 that runs on the mobile device 132 or on the RPT device 122. Such an application may be a dedicated application such as MyAir™ or a web browser that interacts with a website provided by the health or home care provider.

Collected data received from the RPT device 122 or the mobile device 132 is stored and indexed in the database 150 by the data server 312 so as to be uniquely associated with the patient 110 and therefore distinguishable from data collected from other devices in the system 300. In this regard, although only three RPT user systems are illustrated in FIG. 3 for case of explanation, the system 300 may include many more RPT user systems with corresponding RPT devices, sensors, mobile computing devices, and other components. The data server 312 may be configured to calculate summary data for each session from the data received from the RPT device 122. The data server 312 may also be configured to receive data from the mobile device 132, including data entered by the respective patient 110, behavioral data about the user, or qualitative data.

The EMR server 314 contains electronic medical records (EMRs), both specific to the patients of the system and generic to a larger population of patients with similar disorders to the patient 110. An EMR, sometimes referred to as an electronic health record (EHR), typically contains a medical history of a patient, including previous conditions, treatments, co-morbidities, and current status. The EMR server 314 may be located, for example, at a hospital where any of the patients have previously received treatment. The EMR server 314 is configured to transmit EMR data to the data server 312, possibly in response to a query received from the data server 312.

In this example, the HCP server 316 is associated with the health/home care provider (which may be an individual health care professional or an organization) that is responsible for the patient's respiratory therapy. An HCP may also be referred to as a DME or HME (domestic/home medical equipment provider). The HCP server 316 may host a process 332. One function of the HCP server process 332 is to transmit data relating to the patients to the data server 312, possibly in response to a query received from the data server 312.

In some implementations, the data server 312 is configured to communicate with the HCP server 316 to trigger notifications or action recommendations to an agent of the HCP such as a nurse, or to support reporting of various kinds. Details of actions carried out are stored by the data server 312 as part of the engagement data. The HCP server 316 hosts the HCP server process 332 that communicates with the analysis engine 140 and the applications on the mobile device 132.

For example, the HCP server process 332 may provide compliance analysis based on the use of the RPT device 122 in accordance with compliance rules that specify the required usage over a compliance period, such as usage of the device for at least 4 hours per night on 70% of nights during any consecutive 30-day period within the first 90 days of therapy. The summary data post-processing may determine whether the most recent time period is a compliant session by comparing the usage time with the minimum duration from the compliance rule. The results of such post-processing are referred to as “compliance data.” Such compliance data may be used by a health care provider to tailor therapy that may include the RPT device 122 and other mechanisms. Other actors such as payors may use the compliance data to determine whether reimbursement may be made to a patient. Thus, in this example, the process 332 may be part of a diagnosis, compliance and therapy management application accessible through the user device 170 or a workstation associated with a healthcare provider. An example compliance and therapy management application may be the AirView™ application. Alternatively, the compliance prediction may be used to communicate with the RPT device 122 to change the respiratory therapy to the patient in response to the compliance prediction.

As may be appreciated, data in the data server 312, EMR server 314 and HCP server 316 is generally confidential data in relation to the patients in the system. Typically, patients must provide permission to send the confidential data to another party. Such permissions may be required to transfer data between servers 312, 314, and 318 if such servers are operated by different entities.

In this example the machine learning engine 180 is executed by the data server 312 to provide compliance predictions through a machine learning model. The process 322 tracks patients using a respiratory therapy device such as the RPT device 122 in the general patient population and provides compliance data. Additional data may include patient demographic data and contextual data such as the environment. The compliance data combined with usage and user demographic data allows training of the machine learning model to predict compliance for other patients.

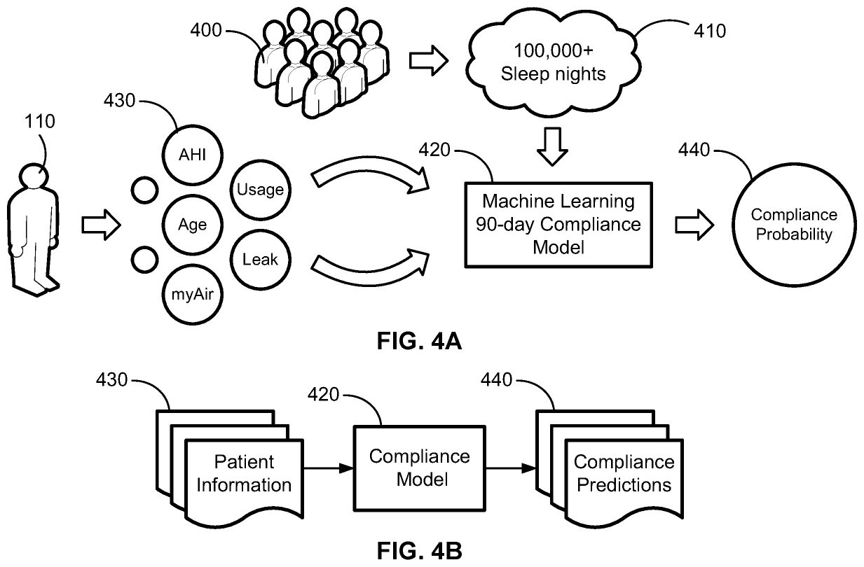

FIG. 4A shows the training and utilization process for the machine learning compliance prediction model of the machine learning engine 180. A general population of patients 400 produces various sets of input data 410. In this example, the input data 410 may include demographic information such as age, operational data from use of a therapy device such as the RPT device 122, usage data of the RPT device 122 and other relevant data correlated with compliance. The input data 410 also includes the compliance results for the general population of patients 400. A machine learning model 420 is trained from the large dataset 410 to provide sufficient confidence in the predictions in relation to the compliance data. Once trained, the machine learning model 420 may accept inputs 430 from individual patients such as the patient 110 to generate a compliance prediction 440.

Once the machine learning model is trained, it may be executed by a server such as the server 312 to provide compliance predictions for users. FIG. 4B shows the process of using a trained compliance prediction model 420. Patient information collected from the patients as well as other associated data such as usage and operational data from the RPT is input to the model 420. Compliance predictions 440 are then output by the trained compliance prediction model 420.

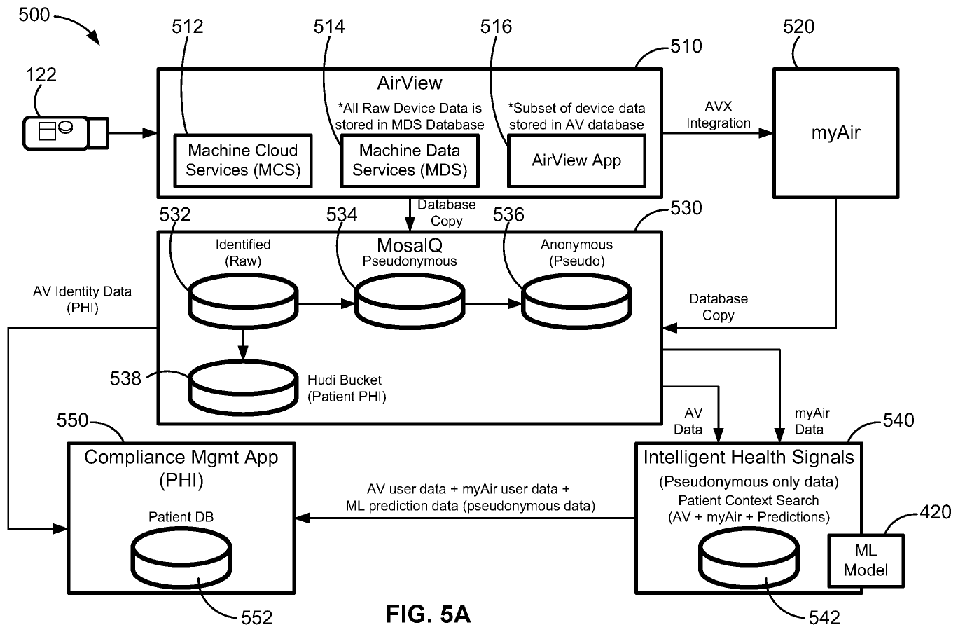

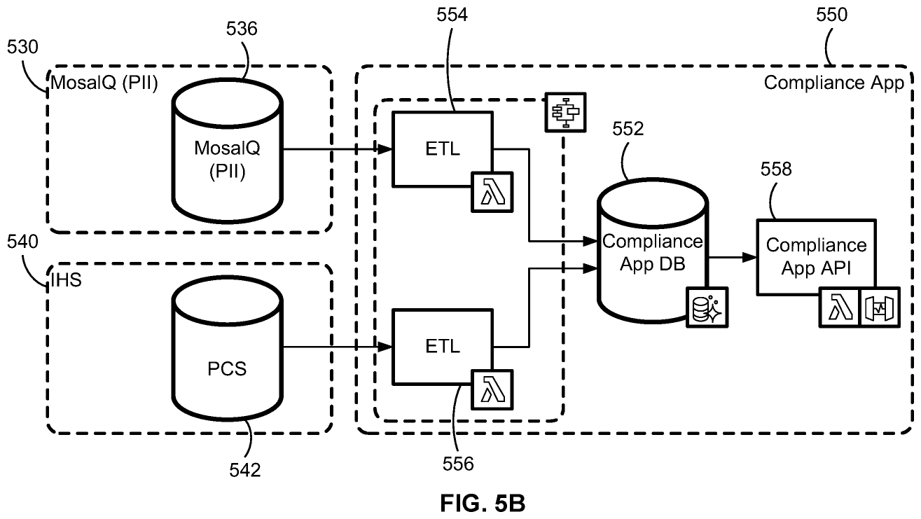

FIG. 5A is a flow diagram 500 of the collection of data for the machine learning model 420 to provide prediction output data. Usage data is produced by RPT devices such as the RPT device 122 associated with a patient such as the patient 110 in FIG. 1. In this example, a healthcare provider system running a diagnosis, compliance and therapy management application 510 such as the AirView™ application collects usage data from the RPT device 122. A personal patient information application 520 such as MyAir™ may receive collected operational data from the RPT device 122 for display to the patient on a user device such as the user device 132 in FIG. 1. The data collected by the applications 510 and 520 is copied by a patient management information system 530. A machine learning engine 540 accesses selected data from the database 542. A compliance management API 550 receives the output from the machine learning routine 540 and provides prediction data.

In this example, the application 510 includes a machine cloud service module 512, a machine data service module 514, and an application 516. The machine cloud service 512 includes APIs to receive data from the RPT devices such as the RPT device 122 over the network 136 in FIG. 1. The machine data service 514 manages a database that stores all raw device data received by the machine cloud service 512. The application 516 is integrated with a user device operated by a healthcare provider such as the user device 170 in FIG. 1. The application 516 accesses a subset of the data from the machine data service 514 for different purposes such as diagnosis of the patient, compliance analysis of the patient, and therapy management of the patient.

The patient management information system 530 includes an identified data repository 532, a pseudonymous data repository 534, and a fully anonymized data repository 536. The identified or raw database 532 is a copy of the databases associated with the database accessed by the application 516 and a database accessed by the application 520. The data from the identified data repository 532 is processed using a pseudonymizing routine to obscure protected health information (PHI) identifiers such as name and address and to merge the data sources together to form the data repository 534. The deidentified data is deposited in a Hadoop Upserts and Incrementals (HUDI) PHI bucket 538 to improve efficiency of pulling data into applications such as a compliance management application. Thus, the same patient is identified in both data sources and combined in the data repository 534. Finally, the pseudonymized PHI is removed entirely using another process to create the anonymized data repository 536. The removal of the pseudonymized PHI also increases security by protecting privacy of the data.

In this example, the machine learning engine 540 is the Intelligent Health Signals (IHS) system developed by ResMed. The machine learning engine 540 includes a database 542 that receives the anonymous data from the anonymized data repository 536 in the form of patient specific data received from the applications 510 and 520. For example, the diagnosis, compliance and therapy management application 510 provides basic patient information like name and address along with device usage whereas the personal application 520 provides more attributes about the patient like age, gender and mask selection and specialized data. In this example, the personal application 520 provides AirView data specific to ResMed. The machine learning engine 540 includes the compliance prediction model 420 that receives the results of a patient context search from the inputs from the diagnosis, compliance and therapy management application and the patient specific data from the personal application 520 and outputs a prediction for the patient that is stored in the database 542. The compliance management application 550 accesses the machine learning model 420 and stores the prediction and patient identity data received from the patient management information system 530 in a compliance database 552. The compliance management application 550 receives the compliance prediction and other anonymized data such as protected health information (PHI) from the machine learning engine 540 and reintroduces the PHI data from the raw database 532. The compliance management application 550 may be accessed by a user application that pulls patient data from the compliance database 552 and presents it to healthcare provider users. In this example, the compliance management application 550 may be a separate component accessible by the diagnosis, compliance and therapy management application 510 or a module of the diagnosis, compliance and therapy management application 510. The compliance management application 550 may send usage data such as filtered data, sorted data, information relating to contacting patients, and the like, back to the patient management information system 530. This increases the potential amount of data for updating the machine learning model 420 and allows for the building of future models around intervention, thereby increasing the overall accuracy of the system's ability to predict compliance of a patient.

Thus, in this example, the system 500 combines identified patient data from a based patient management information system 530 with the machine learning model output data from the patient context search (containing the calculation of non-compliance risk) performed by the machine learning engine 540. In this example, the system 500 may be adapted to use Cloud services such as AWS to provide scalability for users as needed.

In this example, the machine learning model 420 is based on a 90-day compliance prediction model that predicts the probability that a given patient on a specific day of therapy will hit the Center of Medicare & Medicaid Services (CMS) definition of compliance. Specifically, compliance is defined as the patient used the device for at least four hours for 21 non-consecutive days in a 30 day window within the first 90 days of treatment. If a patient hits this criteria at least once in their first 90 days, they have reached compliance and are labeled “compliant.” If a patient does not reach the criteria, they are labeled as “non-complaint.” The example compliance prediction model is used to make a prediction every day for a patient within their first 90-days of treatment. The outputs of this model is available for consumption by downstream applications for both the patient and other actors such as the healthcare provider.

The prediction is termed a “classification” problem, which is a type of supervised learning. The machine learning model 420 is trained on historic data such as that from the patient population 400 and resulting compliance during the first 90 days of treatment. The model then makes predictions based on new patient data. In this example, the machine learning model 420 is trying to predict a binary outcome: non-compliant (0) or compliance (1). The prediction is in the form of a probability between 0 and 1. Thus, a prediction of 0.73 can be interpreted as: “On this day, we believe patient X has a 73% chance of reaching compliance.

The input data, commonly called “features”, are from the Patient Context Service (PCS) database 542 in the Intelligent Health Signals (IHS) machine learning routine 540. In this example, the PCS database 542 sources its data from MyAir™ and AirView™ tables in the non-PHI data lake stored by the patient data system 530. The data may be formatted for any suitable database format such as MOSAIQ. The non-PHI data which has had protected health information removed from the data source (e.g., age, gender, email address, etc). The IHS routine 540 retrieves the raw data from the data lake 536, reformats the raw data to reduce the need for additional pre-processing steps and increase the efficiency of training the model 420. The IHS routine 540 performs operations on the data before saving data to the PCS database 542. An example operation could be: take the average device usage over the last 3 days, and save it as a new feature. The PCS system also generates “targets,” which is what the machine learning model is trying to predict. The targets for the compliance prediction model constitute whether or not a patient reached compliance. For example, a target based on the CMS compliance definition is defined as usage of the device for at least 4 hours per night on 70% of nights during a consecutive 30-day period within the first 90 days of therapy. Some of the features that are in the Patient Context Service database 542 which form inputs to the target prediction include: baseline AHI, duration hours, gender, mask type description, survey reasons for treatment, usage means (e.g., 3, 7, 14, and 28 day means).

The compliance prediction model outputs predictions for all compliance eligible patients in the patient data system 530. This subset of patients is grabbed directly from the database or data lake 536. For example, all patients in their first 90 days or therapy may be selected from the data lake 536. A new forecast of compliance for these new patients is performed by the machine learning model every day over the 90 day period. As a patient is on therapy longer, the patient will generate more usage data features that can be incorporated into the machine learning model. For example, when a patient has been on therapy for n days, a valid feature for that patient would be an aggregate summary of usage over the past n days.

In this example, the machine learning model 420 is trained with historical data features and ground truth compliance targets. The feature data and targets are used to train a model. Internally, the model is storing the functional mapping from features to targets as model coefficients. As the model sees more examples, the model “learns” by updating its internal coefficients. Once the model is trained, new input features are inputs to the fitted model, which returns predicted values. The tech stack used in this example for modeling is sklearn, the most commonly used machine learning package in Python. The model type may be changed. There may thus be a variety of model types such as logistic regression, random forest, etc. The only difference between these models is the internal structure of the model's mapping function. Fundamentally, the types of models that may be predicting compliance are all performing the same function.

Once a compliance prediction model is trained, the compliance prediction model can output predictions based on new patient data. In this example, an additional artifact called shap values is predicted along with the predictions by the compliance prediction model. Shap is a machine learning tool for model explainability. The shap values say how much each feature contributed to the output predictions. This allows a user of the model to interpret the results e.g., “this person had a high predicted compliance because they had low mask leak over the past 7 days” (here “mask_leak_7_days” is a feature). In this example, features for the compliance prediction model may include demographic data such as age and gender, patient response data such as baseline AHI, patient input data such as reasons for treatment obtained by surveys operated by the application executed by the user device 132 in FIG. 1, and usage data such as duration hours, and usage over 3, 7, 14, and 28 day means derived from the operational data collected by the compliance analysis engine 140 from the RPT device 122 in FIG. 1. An example survey may be provided by the personal application on the user device 132 to ask questions relating to reasons for treatment, reactions to the treatment and subjective feeling. A survey could thus ask questions such as “How sleepy do you feel?” and “How is therapy going?” Responses to these questions can be added to the compliance prediction model to improve training in the future.

FIG. 5B is a detailed diagram of the process of the compliance application 550 gathering feature data from the patient information system 530 and the machine learning engine 180. The compliance application 550 includes an extraction, transformation, and loading (ETL) module 554 that combines the data relating to patients from the patient information system 530. Another ELT module 556 receives the output prediction values from the machine learning database 542 of the machine learning engine 540. The ETL modules 554 and 556 send the resulting output data to the compliance application database 552. An API 558 accesses the database to provide the patient data and appropriate compliance prediction data to applications such as the management application 510.

The prediction compliance data may be used in the system 100 in several beneficial ways to improve future compliance, increase efficiency of utilization of resources, and increase treatment for the respiratory ailment. For example, the compliance data may be provided to notify a patient on the user device 132 to further motivate the patient or warn the patient of a possible decrease in probability of compliance. For example, an application on the user device 132 may have a set of rules to play motivational media if a prediction of non-compliance reaches a certain threshold level. Periodically, the training of the machine learning model may be updated with the new patient data and compliance results to improve accuracy of the machine learning model.

The compliance data for a set of patients may be provided to a health care provider allowing the health care provider to prioritize patients by the probability of non-compliance. The compliance prediction data is an objective prediction that eliminates subjective judgement from a healthcare provider. Specific features that contribute to potential non-compliance may be provided as a notification to a health care provider to allow devising strategies to address such features. The probability of non-compliance and when in the 90 day journey it occurs, may be used to determine the best form of intervention by the health care provider and when it is the best time to intervene with the patient. A rules engine may be run as part of the operating routine of the RPT device 122 to adjust the operation of the RPT device 122 to address operational or usage features that are contributing to a prediction of non-compliance.

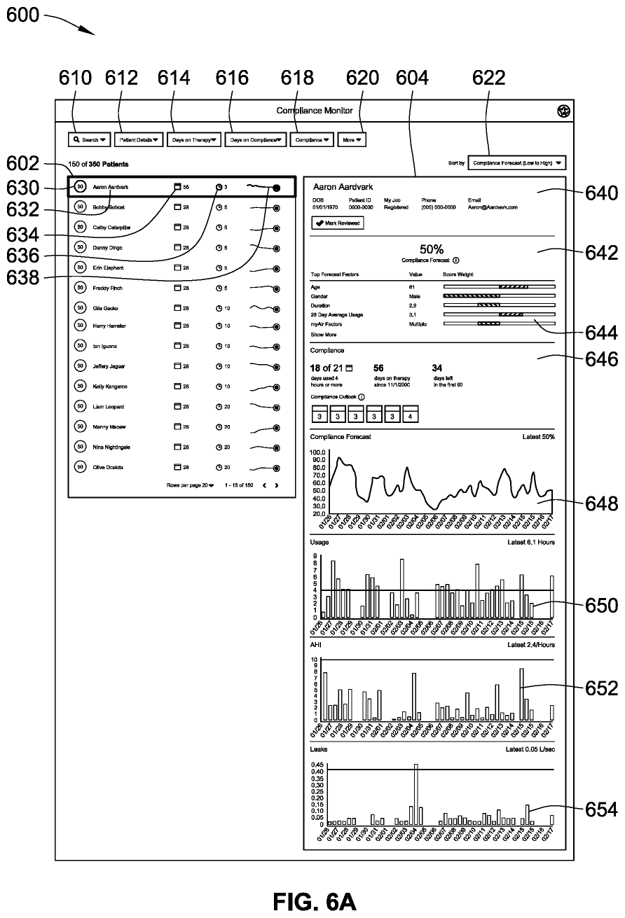

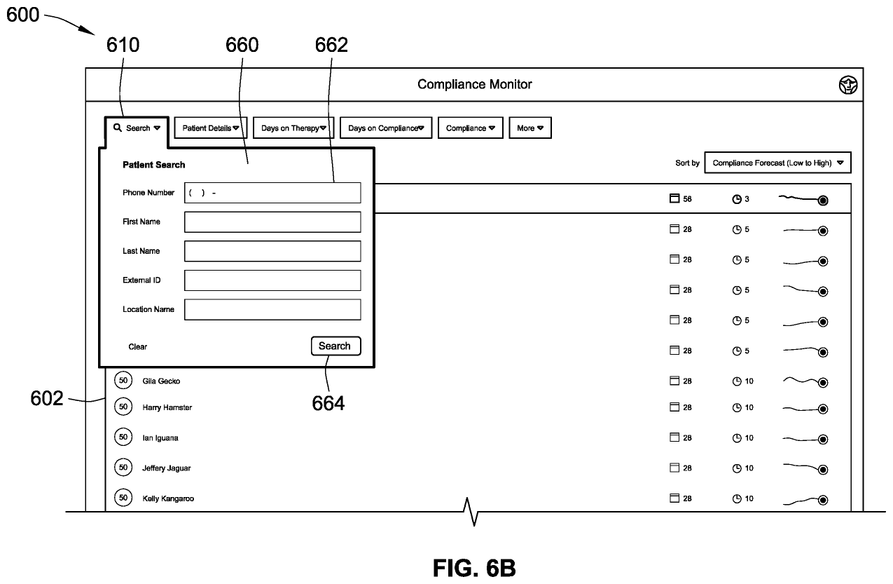

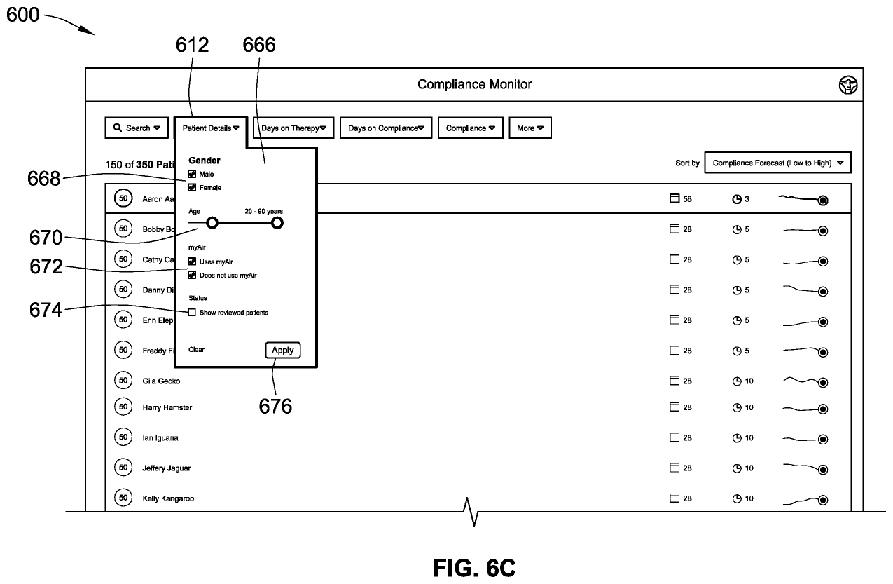

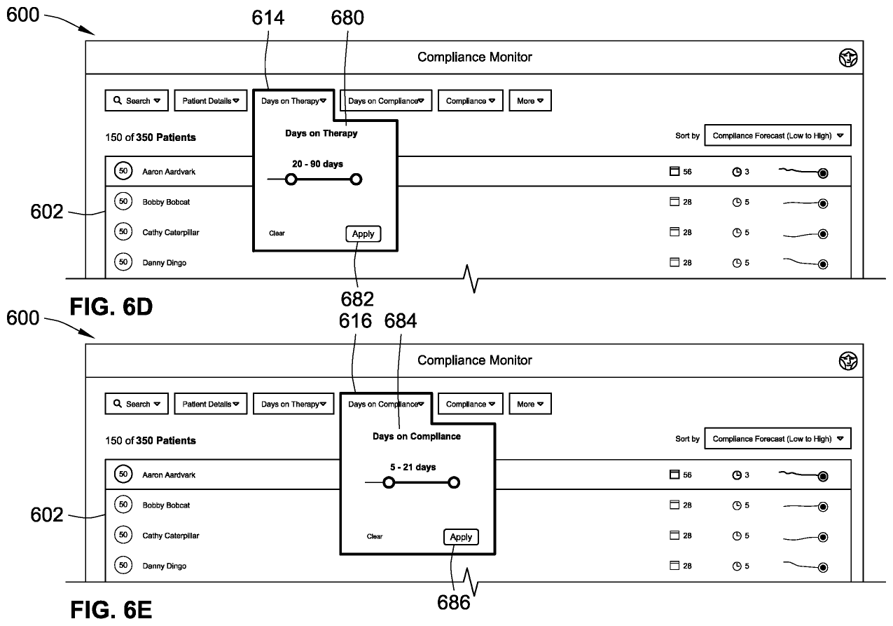

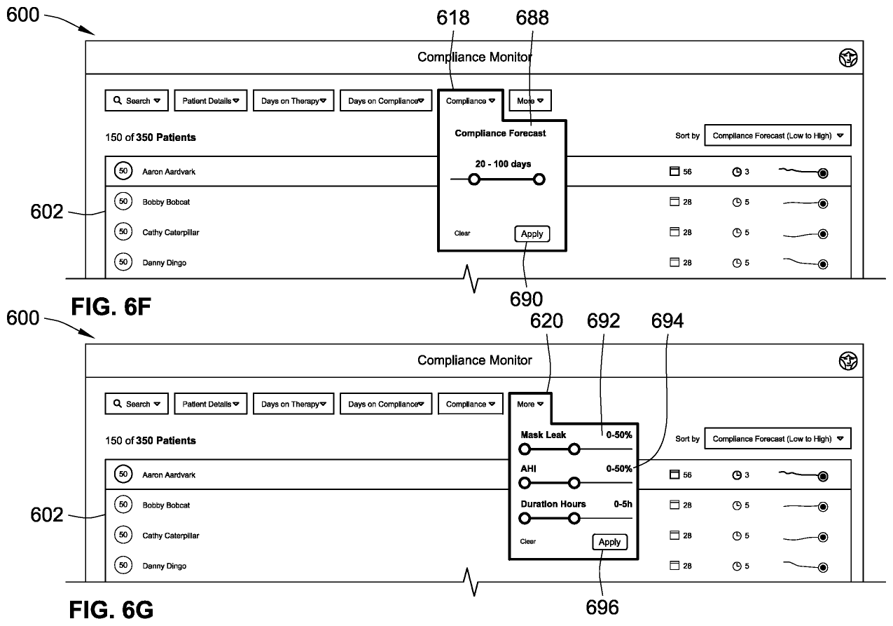

FIG. 6A is an example interface 600 generated by a diagnosis, compliance and therapy management application accessible by a healthcare provider based on the prediction and patient data from the compliance API 558 in FIG. 5B. The example interface 600 may be generated on the display of a workstation of a user device such as the user device 170 used by a healthcare provider. The example interface 600 allows relevant prediction data from the machine learning model to be presented in a useful format to a healthcare provider.

In this example the interface 600 includes a patient summary window 602 that includes a listing of patients associated with the healthcare provider. Each patient listed in the window 602 may be selected and a detailed patient data window 604 may be displayed for the selected patient. The patients displayed in the summary window 602 may be filtered by selecting one of a search tab 610, a patient details tab 612, a days in therapy tab 614, a days to compliance tab 616, a compliance tab 618 and a more tab 620. The default display will list all patients in the patient summary window 602. Each of the patients listed may be ordered by selecting a sort by field 622. In this example, the patients with the lowest compliance forecast are displayed at the top of the list in the window 602. The compliance forecast is derived from the compliance prediction output by the machine learning model 544 in FIG. 5A.

In this example, each of the listings in the patient summary window 602 include a compliance score 630, a name field 632, a days in treatment field 634, a days to complete compliance field 636, and a compliance trend graph 638. The compliance score 630 is the compliance prediction output of the machine learning model based on the specific patient data and usage data. The days in treatment field 634 shows the day the patient is at in the 90 day treatment timeline. The days to complete compliance field 636 shows the number of days that the patient may use the RPT device 122 to complete compliance. The trend graph 638 shows whether the prediction for compliance is increasing or decreasing.

The detailed patient data window 604 includes a patient information field 640, a compliance forecast field 642, a relevant factors field 644, a compliance summary field 646, a compliance forecast graph 648, a usage graph 650, an AHI score graph 652, and a leaks graph 654. The patient information field 640 shows patient information such as the patient name, date of birth, ID number, status for registration of a personal application, phone number, and email. The compliance forecast field 642 shows the predicted percentage of compliance as determined by the machine learning model.

The top forecast factors field 644 shows the input data that most influences the calculation of the compliance prediction score based on the shap value for the data feature. In this example, the most significant five factors may be shown. This number may be adjusted by the healthcare provider or set at a default number. Each of the factors will show the values determined from the patient data and usage data collected by the system 100 in FIG. 1. Each factor will have a corresponding graphic that shows either a color such as green for a positive contribution to the compliance prediction or red for a negative contribution to the compliance prediction. In this example, the positive factors are the age of the patient and the 28 day average usage. The negative factors are the gender, the average duration of the usage, and multiple factors determined by the patient responding to a survey on the application interface on the user device such as the user device 132 in FIG. 1.

The compliance summary field 646 includes background information relating to compliance. In this example, the compliance summary field 646 includes the days where the RPT device 122 was used over a certain threshold level (e.g., 4 hours), the number of days out of the 90 day timeline the patient is on, the days left to complete 90 days, and a compliance outlook. The compliance outlook represents the number of days at over threshold usage that the patient will need to reach compliance.

The compliance forecast graph 648 plots the determined compliance predictions for the patient over a period of time such as over the past three weeks. The usage graph 650 summarizes usage data for the RPT device 122 over the past three weeks and the number of hours per use. The leaks graph 654 plots the leakage data from the interface 124 in FIG. 1 collected from the RPT device 122 charted over the past three weeks.

The Apnea-Hypopnea Index (AHI) score graph 652 shows the determined AHI scores over the last three weeks. The Apnea-Hypopnea Index is an index used to indicate the severity of sleep apnea during a sleep session. The AHI is calculated by dividing the number of apnea and/or hypopnea events experienced by the user during the sleep session by the total number of hours of sleep in the sleep session. The event can be, for example, a pause in breathing that lasts for at least 10 seconds. This pause may be determined by the sensors in the RPT device 122 or by other sensors on wearable devices. An AHI that is less than 5 is considered normal. An AHI that is greater than or equal to 5, but less than 15 is considered indicative of mild sleep apnea. An AHI that is greater than or equal to 15, but less than 30 is considered indicative of moderate sleep apnea. An AHI that is greater than or equal to 30 is considered indicative of severe sleep apnea. In children, an AHI that is greater than 1 is considered abnormal. Sleep apnea can be considered “controlled” when the AHI is normal, or when the AHI is normal or mild. The AHI can also be used in combination with oxygen desaturation levels to indicate the severity of Obstructive Sleep Apnea.

FIG. 6B shows the interface 600 when the specific search tab 610 is selected. The specific search tab 610 causes a popup window 660 to be displayed. The popup window 660 includes search entry fields 662 that allow the user to enter specific values for phone number, first name, last name, ID, and location. Once one of more of the search entry fields 662 are populated, a search button 664 may be selected and the patients that meet the search criteria may be displayed in the patient summary window 602. Other fields such as email may also be presented for search.

FIG. 6C shows the interface 600 when the patient details tab 612 is selected. The patient details tab 612 causes a popup window 666 to be displayed. The popup window 666 has filter controls including a gender selection field 668, an age slider 670, a patient application use field 672, and a reviewed status field 674. The gender field 668 allows the selection of patients of a certain gender. The age slide 670 allows selection of patients between certain ages set by the slider. The patient application use field 672 and the status field allows selection of patients that use the patient application such as MyAir™ and patients that have not been reviewed by the health care provider. Once one of more of the filters are applied, an apply button 676 may be selected and the patients that meet the filtered criteria may be displayed in the patient summary window 602.

FIG. 6D shows the interface 600 when the days in therapy tab 614 is selected. The days in therapy tab 614 causes a popup window 680 to be displayed. The popup window 680 has a days in therapy slider that allows a user to set the range of days in therapy. Once the days in therapy range is selected, an apply button 682 may be selected and the patients that are within the range of days in therapy criteria may be displayed in the patient summary window 602.

FIG. 6E shows the interface 600 when the days in compliance tab 616 is selected. The compliance tab 616 causes a popup window 684 to be displayed. The popup window 686 has a days in compliance slider that allows a user to set the range of days in therapy. Once the days in compliance range is selected, an apply button 686 may be selected and the patients that meet the days in compliance range may be displayed in the patient summary window 602.

FIG. 6F shows the interface 600 when the compliance tab 618 is selected. The compliance tab 618 causes a popup window 688 to be displayed. The popup window 688 has a compliance forecast slider that allows a user to set the range of compliance prediction values. Once the range of compliance prediction values is selected, an apply button 686 may be selected and the patients that meet the range of predicted compliance may be displayed in the patient summary window 602.

FIG. 6G shows the interface 600 when the usage data tab 620 is selected. The usage data tab 620 causes a popup window 692 to be displayed. The popup window 692 has a series of sliders 694 that allow a user to select the percentage of mask leak, the value of AHI score, and the duration in hours as filters. Once the applicable ranges for any or all of mask leak, AHI and duration of use is selected on the sliders 694, an apply button 696 may be selected and the patients that meet the selected values may be displayed in the patient summary window 602.

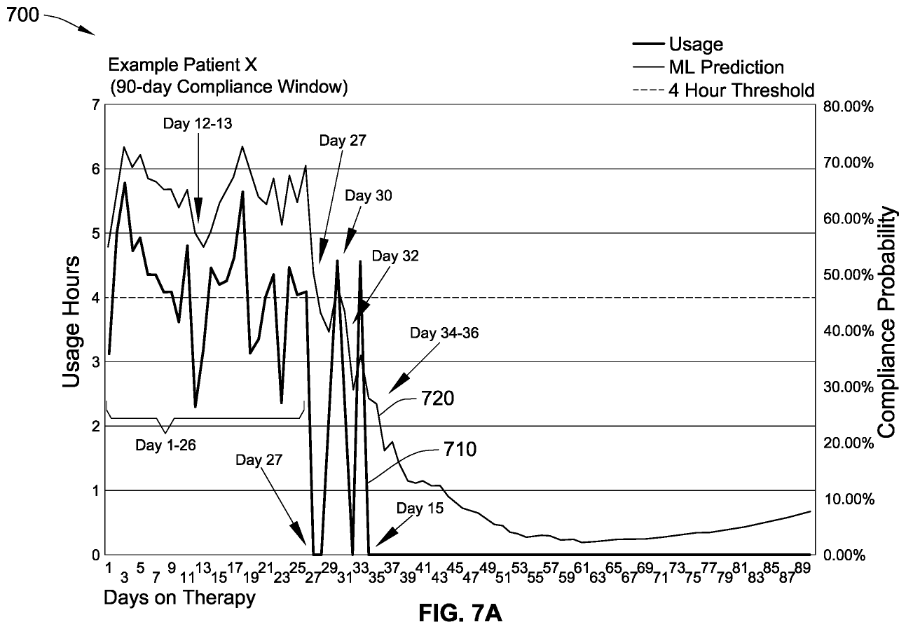

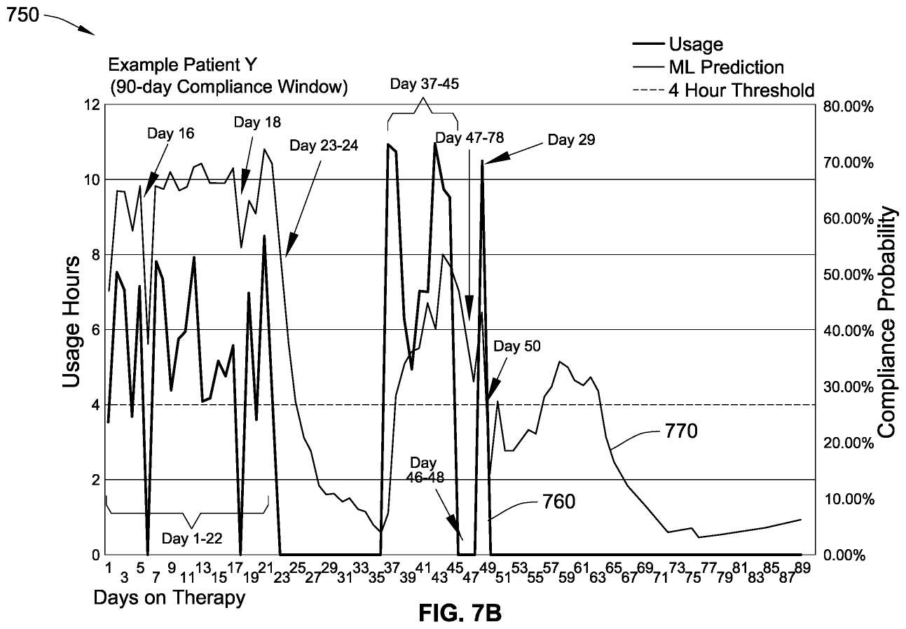

FIGS. 7A-7B depict opportunities for intervention by the health care provider that would otherwise not be obvious with conventional methods of deciding when to intervene. The opportunities arise whenever the probability of compliance dips significantly from the mean ML prediction. For example, the weight assigned to an absent day of therapy during the first week is rated higher than during the second week of therapy because the machine learning model has learned that it is a more important determinant of eventual compliance. Existing methods of determining when to intervene do not have this weighting capability. In this manner, treatment compliance and thus treatment of respiratory ailments may be improved for such patients that are identified.

FIG. 7A shows a graph 700 of an example 90 day compliance prediction model predicting a one day compliance miss. In this example, the graph 700 plots usage hours of an RPT device over the 90 day compliance period. In this example, a four hour period of use on a given day is a threshold level for over 50% compliance. The graph 700 includes a plot of actual usage 710 and a plot of the output of the machine learning compliance prediction model 720.

The plot of actual usage 710 shows that the patient has used the RPT device for the first 26 days straight, but many of the days fall short of the four hour threshold. Day 27 is the first day of not using the RPT device. There are two uses during day 30 and day 33 that are only one day of usage away from compliance, but after day 34, there is no usage during the remaining days.

The machine learning plot 720 is determined by different predictions determined daily over the 90 day period. The machine learning plot 720 is significantly less noisy than the daily usage plot 710 as it accounts for other input factors such as patient demographics, trends, etc.) The shap values that go into the daily computation of compliance probability vary from day to day for each patient. The major features determined by the shap values are determined and displayed as shown in FIG. 6A. Daily usage is usually a major determinant of compliance probability but it is not the only one, which is why the plot 720 is less noisy than the usage plot 710. Since the machine learning plot 720 is a prediction, it allows detection of opportunities for intervention by a healthcare provider. For example, during days 12-13, the prediction of compliance drops by 10% (65% to 55%), during day 27, the prediction of compliance drops by 20% (69% to 49%), during day 32, the prediction of compliance drops by 14% (43% to 29%), and between days 34-36, prediction of compliance drops by 17% (35% to 18%). The periods where drops may be predicted allows the health care provider to intervene with the patient to attempt to boost compliance. Alternatively, the routine may determine sharp compliance drops and automatically attempt to motivate the patient through the patient application.

FIG. 7B shows a graph 750 of an example 90 day compliance prediction model predicting a four day compliance miss. In this example, the graph 750 plots usage hours of an RPT device over the 90 day compliance period. The graph 750 includes a plot of actual usage 760 and a plot of the output of the machine learning compliance prediction model 770.

The plot of actual usage 770 shows that the patient has used the RPT device for the frequently during days 1-22 with 2 days of no usage. Thus, the patient is only 4 days away from compliance. However, during days 23-36, there is no usage for 14 consecutive days. During days 37-45, there is frequent and high usage, making the patient only 7 days from compliance. This is followed by a period of no usage during days 46-49 and one last day of usage on day 49

The machine learning plot 770 is determined by different predictions determined daily over the 90 day period. Since the machine learning plot 770 is a prediction, it allows detection of opportunities for intervention by a healthcare provider. For example, during day 6, prediction of compliance drops by 35% (82% to 47%), during day 18, the prediction of compliance drops by 18% (86% to 68%), during days 23-24, the prediction of compliance drops by 40% (87% to 47%), during days 47-48, the prediction of compliance drops by 10% (59% to 38%), and during day 50, prediction of compliance drops by 37% (54% to 17%).

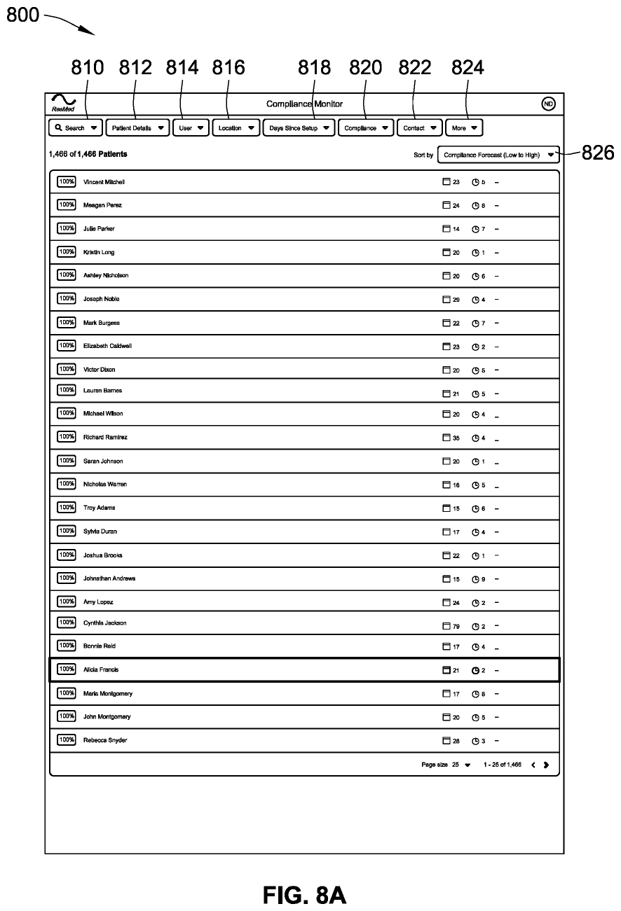

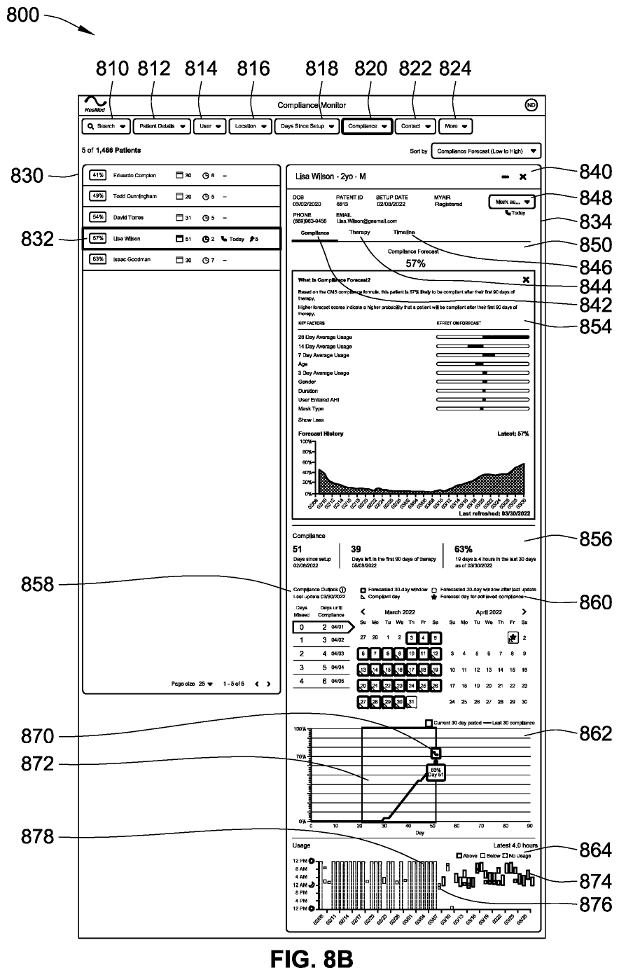

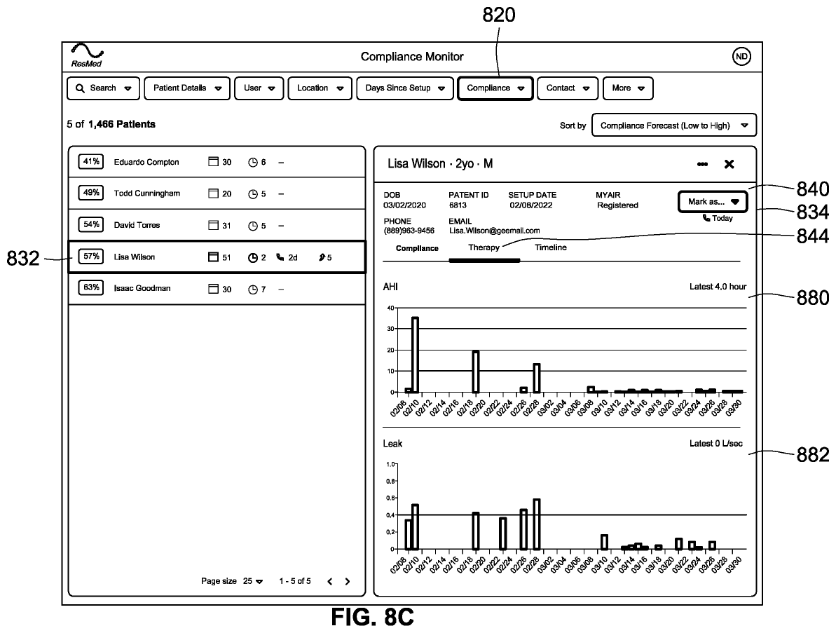

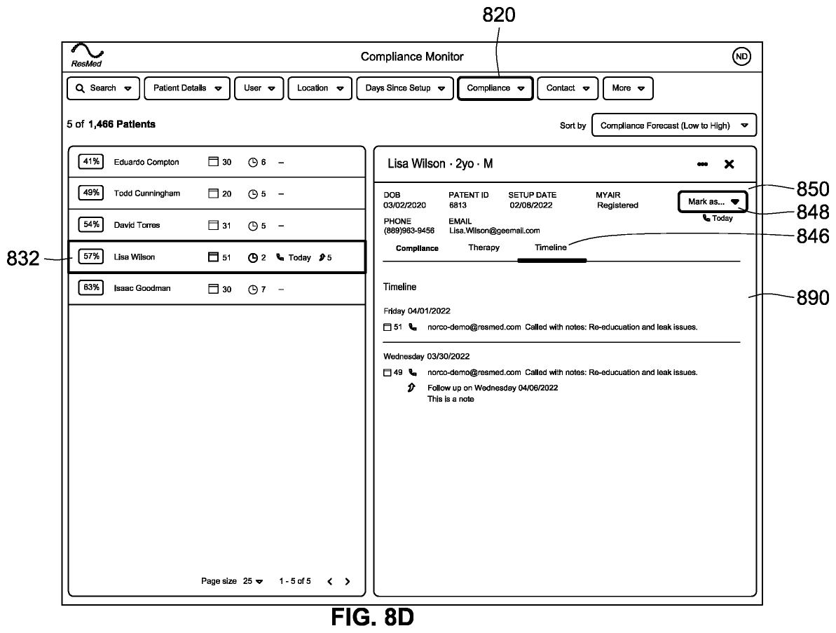

FIG. 8A is another example interface 800 generated by a diagnosis, compliance and therapy management application accessible by a healthcare provider based on the prediction and patient data from the compliance API 558 in FIG. 5B. The example interface 800 may be generated on the display of a workstation of a user device such as the user device 170 used by an operator of a healthcare provider. Similar to the interface 600 in FIGS. 6A-6G, the example interface 800 allows relevant prediction data from the machine learning model to be presented in a useful format to a healthcare provider.

Identical to the example interface 600 described above, the interface 800 includes a patient summary window 802 that includes a listing of patients associated with the healthcare provider. The window 802 has similar information to the window 602 described in reference to FIG. 6A. Each patient listed in the window 802 may be selected and a detailed patient data window may be displayed for the selected patient. The patients displayed in the summary window 802 may be filtered by selecting one of a search tab 810, a patient details tab 812, a users tab 814, a location tab 816, a days since setup tab 818, a compliance tab 820, a contact tab 822, and a more tab 824. The search tab 810, patient details tab 812, days since setup tab 818, and compliance tab 820 have similar functions as their counterpart tabs 610, 612, 616, and 618 in FIG. 6A. Selecting the users tab 814 allows an operator to filter down to patients managed by a specific clinical user (including themselves). The location tab 816 allows an operator to filter down to patients served by different physical locations operated by a health care provider or other entity.

The contact tab 822 allows the display of a window that shows filters that search for patients based on the last time they were contacted by someone in the provider. In this example, an operator records patients as contacted via the application when contact attempts are made. In this example, the window allows the patients to be filtered via selection of one of a set of contact buttons. The contact buttons include an all patients button that causes all patients to be displayed. Another not contacted button allows display of a list of patients who have never been contacted by anyone in the health provider organization. A contacted button when selected allows the operator to use a slider to select a date range. The contacted button then will display patients who have been contacted within the specified date range from the slide. The start date for the date range is the current date. Moving the slider to 30+ will list all patients who were contacted more than 30 days ago.

The default display will list all patients in the patient summary window 802. Each of the patients listed may be ordered by selecting a sort by field 826. In this example, the patients with the lowest compliance forecast are displayed at the top of the list in the window 802. The compliance forecast is derived from the compliance prediction output by the machine learning model 544 in FIG. 5A. The patients may be sorted by other criteria such as by highest compliance forecast, as well as high to low or low to high days since setup, days to compliance, days since contact, and days to follow-up.

In this example, each of the listings in the patient summary window 802 include the current compliance forecast prediction, a name field, a days in treatment field, and a days to complete compliance field that are identical to their counterparts in the interface 600 in FIG. 6A.

FIG. 8B shows the interface 800 when the compliance tab 820 is selected. The compliance tab 820 displays a compliance slider similar to that shown in FIG. 6A that allows a user view compliance and usage related information to a specific patient. Any of the patients listed in the filtered patient summary window 830 such as a selected listing 832 may be highlighted. Once a listing of a patient is selected, the listing that includes a symbol that indicates a type of contact as well as the time since the last contact with the patient. In this example, the symbols represent a phone message, an email message, or an actual completed call to the patient.

A patient summary window 834 is displayed on the interface 800 when a specific patient listing such as the patient listing 832 is selected. The patient summary window 834 includes a patient information field 840 and a set of patient data options including a compliance option 842, a therapy option 844, and a timeline option 846.

In this example, the patient information field 840 shows the date of birth, patient ID, setup date, status, phone and email. The patient information field also includes a mark as contacted button 848. The mark as contacted button 848 launches a patient contact window. In this example, the operator may select different contact options that record the particular contact with the patient. The options may include a called option that marks the patient as contacted by phone; a left voicemail that marks leaving a voicemail for the patient; and a sent Email option that marks the patient as contacted via email. The contact field on the selected listing 842 is updated when a particular option is selected. The contact window also allows the operator to view the contact history to the patient that shows the outreach history and the method of contact to the patient. The contact window may also be directly linked to different applications that allow an operator automatically contact a listed patient. For example, an automatic dialing API may employ phone data to place a call to the patient or an email application may compose an email.

In this example, the compliance option 842 is selected that shows a compliance forecast field 850. The compliance forecast field 850 includes a compliance forecast 852 that lists the resulting calculation of the listed patient's likelihood of becoming compliant within the first 90 days of therapy based on the CMS compliance rule that is determined according to the process described herein. In this example, the compliance forecast field 852 also includes a what is compliance forecast area 854 that is a field similar to the forecast factors field 644 in FIG. 6A. The what is compliance forecast area 854 thus shows a graph that displays the factors that either positively or negatively influence the compliance forecast. The what is compliance forecast area 854 also shows a forecast history graph over a specified period of time such as thirty days.

The compliance forecast field 850 also includes a compliance summary field 856, a compliance outlook section 858, a calendar 860, a compliance graph 862, and a usage graph 864. The compliance summary field 856 includes background information relating to compliance. In this example, the compliance summary field 856 includes the days that the RPT device 122 has been set up, the days left to complete 90 days of therapy, and a percentage of the days and number of days since setup that the RPT device 122 has been used greater than 4 hours.

The compliance outlook section 458 lists a table showing the days missed for the patient and the number of days and the date until the patient reaches compliance. The compliance outlook section 858 and the calendar 860 allow an operator understand the various scenarios that can affect the patient's ability to achieve compliance if this patient misses one or more days of compliant usage per the CMS rules.

The calendar 860 shows a two month window that includes days with a forecasted 30 day window in one gray shade and days with a forecasted 30 day window after the last update of data for the patient in another gray shade. Each day that the patient was compliant with the treatment is marked with a triangle. A star symbol designates the forecasted day for achieved compliance. Of course other indicators and shapes may be used with the calendar 860 to show time for compliance.

An operator can click the rows in the compliance outlook section 858 to view projected 30-day compliance window scenarios on the calendar 860. As the 30-day compliance window moves, the total number of days needed to achieve compliance will increase if the patient does not use their device. For example, if the second row is selected in the compliance outlook section 858, the calendar will display different shaded days and the star symbol will be moved two days.

The compliance graph 862 is a compliance percent history chart plotted for the first 90 days that allows a user to view how a specific outreach event impacts patient compliance. The compliance graph 862 includes a plot that shows the predicted percent of compliance during a specific day. Contact events such as a phone message, an email, or a successful phone conversation are designated by a contact symbol 870 on the plot. Each type of contact has a specific symbol to show the type of contact attempted to the patient. A current 30 day period is highlighted by a shaded section 872.

The usage graph 864 includes bars that represent usage over a period of the first 90 days of therapy plotted against the times where the usage occurred. Certain bars 874 show usage periods over four hours and may be colored green. Other bars 876 show usage periods under four hours and may be colored red. Days without usage may be designed by hollow bars 878. The fragmented usage graph 864 allows a user to quickly view sessions and view a count of mask on/off events to provide further context for patient conversations.

FIG. 8C shows the interface 800 when the compliance tab 820 is activated and a selected listing such as the listing 832 is selected. In this example, the therapy option 844 has been selected in the displayed patient summary window 834. When the therapy option 844 is selected, an Apnea-Hypopnea Index (AHI) score graph 880 and a leaks graph 882 is displayed for the selected patient in the listing 832. In this example, the AHI graph 880 is similar to the score graph 652 in FIG. 6A and shows the determined AHI scores over the last three weeks. The leaks graph 882 plots the leakage data from the interface 124 in FIG. 1 collected from the RPT device 122 charted over the past three weeks for the selected patient.

FIG. 8D shows the interface 800 when the compliance tab 820 is activated and a selected listing such as the listing 832 is selected. In this example, the timeline option 846 has been selected in the displayed patient summary window 834. When the timeline option 846 is selected, a timeline 890 of events relating to the selected patient is displayed. The timeline 890 includes events that are listed by date, days after the commencement of the compliance period, contacts with the patient, follow-up reminders, and notes that have been marked via the mark as button 848.

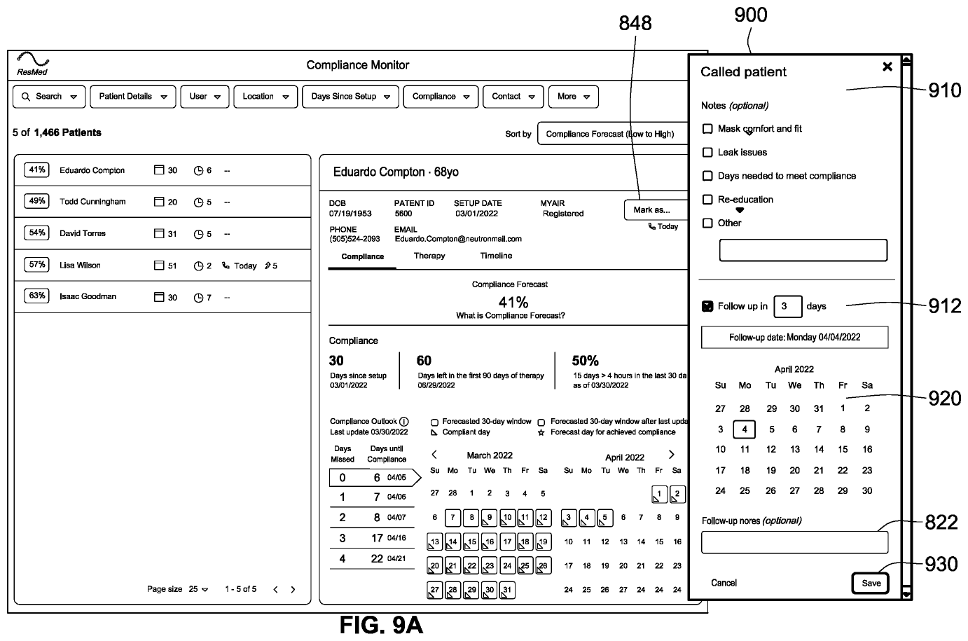

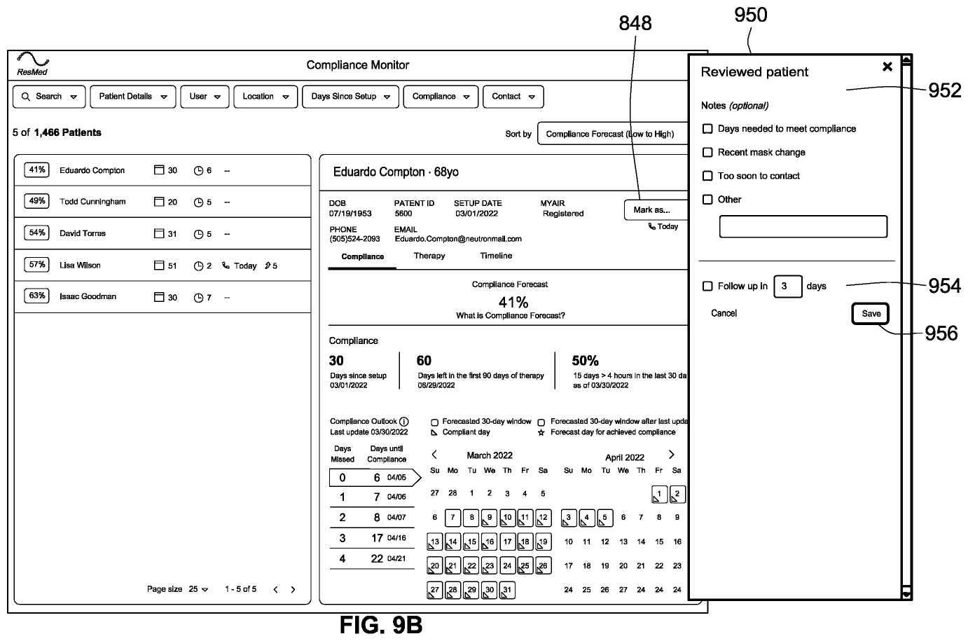

FIG. 9A shows a contact and follow up pop-up window 900 that is displayed by selecting one of the contact options displayed when the mark as button 848 is selected. In this example, the options include a called option, a left voicemail option, and an emailed option. In this example, the popup window 900 is activated by selecting the called option. The popup window 900 has a notes section 910 that includes check boxes with common notes such as mask comfort and fit, leak issues, days needed to meet compliance, and re-education for communication to the patient. An other field allows other notes to be entered. A follow up field 912 is displayed that may be selected to indicate follow-up with the patient after a selected number of days. The follow up field 912 includes the specific date listed of the follow-up as well as a calendar 920 that shows the follow up date. A follow-up notes field 922 allows the user to enter additional notes for the follow-up. A save button 930 allows the user to save the follow-up data from the window 930.

FIG. 9B shows a reviewed patient window 950 that is displayed by selecting the reviewed option in the marked as button 848. The popup window 950 has a notes section 952 that includes check boxes with common notes for a reviewed patient such as days needed to meet compliance, recent mask change, and too soon to contact. An other field allows other notes to be entered. A follow up field 954 is displayed that may be selected to indicate follow-up with the patient after a selected number of days. A save button 956 allows the user to save the patient reviewed data from the window 950.

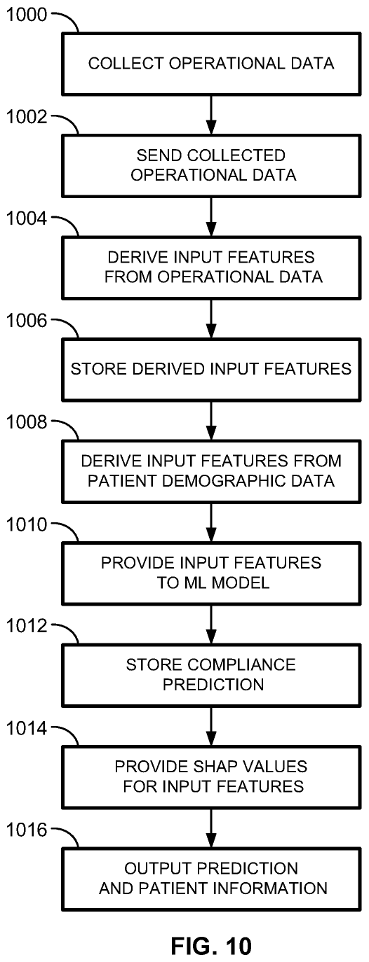

FIG. 10 shows a flow diagram for the data collection and prediction routine performed by the system 100. The flow diagram in FIG. 10 is representative of an example routine implementable by machine-readable instructions for the compliance analysis engine 140 to collect data for determining health conditions for the patient 110 in FIG. 1. In this example, the machine-readable instructions comprise an algorithm for execution by (a) a processor; (b) a controller; and/or (c) one or more other suitable processing device(s). The algorithm may be embodied in software stored on tangible media such as flash memory, CD-ROM, floppy disk, hard drive, solid-state drive, digital video (versatile) disk (DVD), or other memory devices. However, persons of ordinary skill in the art will readily appreciate that the entire algorithm and/or parts thereof can alternatively be executed by a device other than a processor and/or embodied in firmware or dedicated hardware in a well-known manner (e.g., it may be implemented by an application-specific integrated circuit (ASIC), a programmable logic device (PLD), a field-programmable logic device (FPLD), a field-programmable gate array (FPGA), discrete logic, etc.). For example, any or all of the components of the interfaces can be implemented by software, hardware, and/or firmware. Also, some or all of the machine-readable instructions represented by the flowcharts may be implemented manually. Further, although the example algorithm is described with reference to the flowchart illustrated in FIG. 10, persons of ordinary skill in the art will readily appreciate that many other methods of implementing the example machine-readable instructions may alternatively be used. For example, the order of execution of the blocks may be changed, and/or some of the blocks described may be changed, eliminated, or combined.

The system first monitors operational data collected from the RPT device 122 (900). In this example, the compliance analysis engine 140 collects the operational data from the sensors on the RPT device 122 and assigns time stamps and patient identification information. The operational data is sent to the compliance analysis engine 140 via the network 136 in FIG. 1 (1002). The compliance analysis engine 140 derives certain input features from the collected operational data (1004). These input features may include usage duration, leak data, and type of mask. The input features are stored in the machine learning database 452 in FIG. 4A (1006). The compliance analysis engine 140 derives input features related to patient demographics such as age and gender from the external database 160 in FIG. 1 (1008). The input features are then input to the machine learning model (1010).

The output compliance prediction is stored (1012). A shap value is determined for each of the input features (1014). The resulting prediction and patient information from the database 160 is output by the API for transmission to other applications in the system 100 (1016).

As used in this application, the terms “component,” “module,” “system,” or the like, generally refer to a computer-related entity, either hardware (e.g., a circuit), a combination of hardware and software, software, or an entity related to an operational machine with one or more specific functionalities. For example, a component may be, but is not limited to being, a process running on a processor (e.g., digital signal processor), a processor, an object, an executable, a thread of execution, a program, and/or a computer. By way of illustration, both an application running on a controller, as well as the controller, can be a component. One or more components may reside within a process and/or thread of execution, and a component may be localized on one computer and/or distributed between two or more computers. Further, a “device” can come in the form of specially designed hardware; generalized hardware made specialized by the execution of software thereon that enables the hardware to perform specific functions; software stored on a computer-readable medium; or a combination thereof.

The terminology used herein is for the purpose of describing particular embodiments only and is not intended to be limiting of the invention. As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof, are used in either the detailed description and/or the claims, such terms are intended to be inclusive in a manner similar to the term “comprising.”

Unless otherwise defined, all terms (including technical and scientific terms) used herein have the same meaning as commonly understood by one of ordinary skill in the art. Furthermore, terms, such as those defined in commonly used dictionaries, should be interpreted as having a meaning that is consistent with their meaning in the context of the relevant art, and will not be interpreted in an idealized or overly formal sense unless expressly so defined herein.

One or more elements or aspects or steps, or any portion(s) thereof, from one or more of any of claims 1 to 58 below can be combined with one or more elements or aspects or steps, or any portion(s) thereof, from one or more of any of the other claims 1 to 58 or combinations thereof, to form one or more additional implementations and/or claims of the present disclosure.

While various embodiments of the present invention have been described above, it should be understood that they have been presented by way of example only, and not limitation. Although the invention has been illustrated and described with respect to one or more implementations, equivalent alterations and modifications will occur or be known to others skilled in the art upon the reading and understanding of this specification and the annexed drawings. In addition, while a particular feature of the invention may have been disclosed with respect to only one of several implementations, such feature may be combined with one or more other features of the other implementations as may be desired and advantageous for any given or particular application. Thus, the breadth and scope of the present invention should not be limited by any of the above-described embodiments. Rather, the scope of the invention should be defined in accordance with the following claims and their equivalents.

1. A method of predicting compliance with a respiratory treatment regimen for a patient, the method comprising:
collecting operational data from a respiratory pressure therapy device;
collecting demographic data of the patient; and
predicting compliance with the treatment based on inputting operational data and demographic data to a machine learning compliance prediction model having a compliance prediction output, wherein the machine learning model is trained from the operational data and demographic data of a population of patients using respiratory pressure therapy devices.

2. The method of claim 1, further comprising sending the compliance data to a user device operated by a health care provider associated with the patient or sending the compliance data to a user device operated by the patient.

3-4. (canceled)

5. The method of claim 1, further comprising classifying the patient based on a plurality of classifications of the population of patients wherein the machine learning compliance prediction model includes an output based on the classification of the patient.

6. (canceled)

7. The method of claim 1, wherein the machine learning compliance prediction model outputs the prediction each day of the treatment regimen having a predetermined period of time.

8. The method of claim 1, further comprising communicating with the respiratory pressure therapy device to change the respiratory therapy to the patient in response to the compliance prediction.

9-10. (canceled)

11. The method of claim 1, further comprising collecting physiological data from a health monitor, wherein the collected physiological data is input to the machine learning compliance prediction model to determine the prediction.

12. The method of claim 11, wherein the health monitor includes at least one sensor, the at least one sensor selected from one of the group of an audio sensor, a heart rate sensor, a respiratory sensor, a ECG sensor, a photoplethysmography (PPG) sensor, an infrared sensor, an activity sensor, a radio frequency sensor, a SONAR sensor, an optical sensor, a doppler radar motion sensor, a thermometer, an impedance sensor, a piezoelectric sensor, a photoelectric sensor, or a strain gauge sensor.

13. (canceled)

14. The method of claim 1, wherein the machine learning compliance prediction model analyzes one of environmental data related to the patient in determining the prediction or demographic data related to the patient in determining the prediction.

15. The method of claim 1, wherein the machine learning compliance prediction model analyzes demographic data related to the patient in determining the prediction.

16. The method of claim 1, further comprising providing a shap value for each type of usage data and demographic data.

17. The method of claim 1, wherein the collected operation data is used to derive duration of usage, leak data, AHI, and mask type.

18. (canceled)

19. The method of claim 1, further comprising collecting patient input data from a survey, and wherein the machine learning compliance prediction model analyzes the patient input data in determining the prediction.

20. The method of claim 1, further comprising displaying the compliance prediction of the patient in a calendar showing the period of treatment.

21. (canceled)

22. The method of claim 20, wherein the calendar shows past days of compliance and the end of a projected compliance period based on the compliance prediction.

23. (canceled)

24. The method of claim 19, further comprising displaying information relating to a last contact attempt with the patient.

25. (canceled)

26. A non-transitory computer program product comprising instructions which, when executed by a computer, cause the computer to carry out:
collecting operational data from a respiratory pressure therapy device;
collecting demographic data of the patient; and
predicting compliance with the treatment based on inputting operational data and demographic data to a machine learning compliance prediction model having a compliance prediction output, wherein the machine learning model is trained from the operational data and demographic data of a population of patients using respiratory pressure therapy devices.

27. (canceled)

28. A system to predict compliance of a patient with a respiratory treatment regimen, the system comprising:
a respiratory pressure therapy device including a transmitter and an air control device to provide air flow based respiratory therapy to a patient, the respiratory pressure therapy device collecting operational data and transmitting the collected operational data;
a patient database storing demographic data associated with the patient;
a network receiving the collected operational data from the respiratory pressure therapy device and the demographic data from the database; and
a compliance analysis engine coupled to the network, the compliance analysis engine inputting the collected operational data and demographic data to a compliance prediction model trained on a large patient population dataset including operational and demographic data and resulting compliance, wherein the compliance prediction model outputs a prediction of compliance for the patient for using the respiratory pressure therapy device.

29-46. (canceled)

47. The system of claim 28, further comprising a display coupled to the compliance analysis engine, wherein the display displays the compliance prediction of the patient.

48. The system of claim 47, wherein the compliance prediction is displayed in a calendar showing the period of treatment, wherein the calendar shows past days of compliance and the end of a projected compliance period based on the compliance prediction.

49-52. (canceled)

53. A method of training a compliance prediction model for outputting a prediction of compliance for a patient with a respiratory treatment regimen, the method comprising:
collecting operational data from a population of patients each using a respiratory pressure therapy device;
collecting demographic data from the population of patients;
determining compliance data from the population of patients based on the operational data;
selecting a set of input features based on the collected operational and demographic data; and
training a compliance prediction model with the collected operational and demographic data selected in the set of input features and the corresponding compliance data to output a compliance prediction.

54-58. (canceled)
